(19) United States
(12) Patent Application Publication
(10) Pub. No.: US 2021/0069218 A1
(43) Pub. Date: Mar. 11, 2021

(54) METHODS OF CONTROLLING TUMOR BIOENERGETICS NETWORKS

(71) Applicant: The Wistar Institute of Anatomy and Biology, Philadelphia, PA (US)

(72) Inventors: Young Chan Chae, Bryn Mawr, PA (US); Dario C. Altieri, Philadelphia, PA (US)

(21) Appl. No.: 16/833,144

(22) Filed: Mar. 27, 2020

Related U.S. Application Data
(63) Continuation of application No. 14/378,691, filed on Aug. 14, 2014, now abandoned.
(60) Provisional application No. 61/697,434, filed on Sep. 6, 2012.
(60) Provisional application No. 61/598,637, filed on Feb. 14, 2012.

(51) Int. Cl. A61K 31/675; A61K 45/06; A61K 31/4706; A61K 31/519; A61K 31/19; A61K 31/7004; C12Q 1/6886; G01N 33/574

(52) U.S. Cl. CPC A61K 31/675; A61K 45/06; A61K 31/4706; A61K 31/519; G01N 2333/4703; A61K 31/7004; C12Q 1/6886; G01N 33/57496; C12Q 2600/158; A61K 31/19

(57) ABSTRACT

Methods of stimulating anti-tumor activity in a subject with cancer are provided. The methods include administering to a subject in need thereof a low dose low dosage of a composition comprising a molecule that inhibits Hsp90 linked to a mitochondria-penetrating moiety; and administering to the subject an effective amount of an inhibitor of autophagy or glycolysis.

### STATEMENT OF GOVERNMENT INTEREST

This invention was made with government support under Grant Nos. 5P01CA140043-03, 7R01CA078810-14, 5R37HL054131-20, and CA11805 awarded by the National Institutes of Health. The government has certain rights in the invention.

### BACKGROUND

Although targeted cancer therapy is feasible, and has produced, in some cases, good clinical responses, target-centric drug discovery has generated many hopeful agents that provided minimal or no gains when tested in the clinic. The high rate of failure may be due to the extreme heterogeneity of even seemingly identical tumors, carrying hundreds of mutated, amplified, or deregulated genes. Such complexity makes it difficult to identify a single, driving, signaling pathway suitable for therapeutic intervention. Thus, biology tools are now being used to model cancer pathways as globally interconnected networks. This information can be exploited for novel pathway-oriented drug discovery, with the goal of identifying inhibitors of nodal proteins, i.e., molecules that integrate multiple signaling subnetworks. Such network inhibitors may be best suited to simultaneously disable multiple mechanisms of tumor maintenance instead of a single gene and thus overcome the genetic and molecular heterogeneity of progressive disease.

Heat Shock Protein-90 (Hsp90) chaperones oversee protein folding quality control in every organism. This process is essential for cellular homeostasis, buffering proteotoxic stress, and enabling cells to continuously adapt to changes in their internal and external milieus. Hsp90 plasticity has been traditionally linked to the diversity of its ‘client proteins’, molecules implicated in multiple facets of cellular maintenance and that require the chaperone ATPase activity for proper folding, maturation, and subcellular trafficking. However, successful adaptation must also encompass fine-tuning of bioenergetics, nutrient-sensing and stress response signaling networks, including autophagy, and the role of Hsp90 in these pathways has remained unexplored.

### SUMMARY

The present invention is based in part on the inventor's discovery that mitochondrial, but not cytosolic, Hsp90s are required for tumor bioenergetics, and connect to a global nutrient-sensing signaling network exploited for tumor cell survival in humans.

In one aspect, the invention provides a method of stimulating anti-tumor activity in a subject with cancer. The method comprises administering to a subject in need thereof a low dosage of a composition comprising a molecule that inhibits Hsp90 linked to a mitochondria-penetrating moiety; and administering to the subject an effective amount of an inhibitor of autophagy or glycolysis.

In another aspect, the method comprises administering to a subject in need thereof a low dosage of a composition comprising a molecule that inhibits TRAP-1 linked to a mitochondria-penetrating moiety; and administering to the subject an effective amount of an inhibitor of autophagy or glycolysis.

In another aspect, the invention provides a method of selectively inhibiting glucose consumption in a tumor cell, the method comprising administering a low dosage of Gamitrinib, one of a class of geldanamycin (GA)-derived mitochondrial matrix inhibitors, as defined below. In one embodiment, the method further comprises administering an effective amount of an inhibitor of glycolysis or autophagy.

In another aspect, the invention provides Gamitrinib and an inhibitor of glycolysis or autophagy for use in stimulating anti-tumor activity.

In another aspect, the invention provides Gamitrinib and an inhibitor of glycolysis or autophagy for use in treating cancer.

In another aspect, the invention provides a method of stimulating anti-tumor activity in a subject with cancer comprising decreasing the amount of or activity of Grp78 in the subject. See, e.g., Example 7.

In another aspect, the invention provides a method of inhibiting tumor cell production in a subject with cancer comprising decreasing the amount of or activity of Grp78 in the subject. See, e.g., Example 7.

In another aspect, the invention provides a method of stimulating anti-tumor activity in a subject with cancer comprising decreasing the amount of or activity of atg5 in the subject.

In another aspect, the invention provides a method of stimulating anti-tumor activity in a subject with cancer comprising decreasing the amount of or activity of LKB-1 in the subject.

In another aspect, the invention provides a method of stimulating anti-tumor activity in a subject with cancer comprising decreasing the amount of or activity of hexokinase II in the subject. In yet another aspect, the method further comprises administering a low dosage of Gamitrinib.

In another aspect, the invention provides a method for diagnosing a proliferative disease or disorder, e.g., cancer, in a subject. The method includes measuring the level of expression or activity of Grp78 in a biological sample from a mammalian subject, preferably a human subject. When compared to the level of expression or activity of Grp78 in a healthy mammalian subject, an increased level of expression or activity is an indication of a diagnosis of proliferative disease or disorder.

In another aspect, the invention provides a diagnostic method for a proliferative disease or disorder. The method involves measuring the level of expression or activity of Grp78 and at least one additional biomarker in the above-noted biological sample. The combined changes in expression or activity of Grp78 and the additional biomarker from their respective levels of expression or activity in a healthy mammalian subject is an indication or differential indication of a diagnosis of proliferative disease or disorder.

In another aspect, the invention provides a method for monitoring progression of proliferative disease or disorder in a mammalian subject suffering from that disorder. In this method the level of expression or activity of Grp78 in a biological sample from a mammalian subject having proliferative disease or disorder is measured and compared to the level of expression or activity of Grp78 of a temporally earlier biological sample of the same subject.

In another aspect, the invention provides a method of predicting duration of survival in a lung cancer patient. In this method, the level of expression or activity of Grp78 in a biological sample from a mammalian subject having proliferative disease or disorder is measured and compared to the level of expression or activity of Grp78 of control. An level of Grp78 higher than that of the control indicates a likelihood of a shorter overall survival compared to lung cancer patients with low to undetectable Grp78.

### BRIEF DESCRIPTION OF THE DRAWINGS

FIGS. 1a-1j demonstrate regulation of tumor bioenergetics by mitochondrial Hsp90s. (a) FIG. 1a is line graph showing percent ATP in the indicated human tumor cell types, representative of cancers of the breast (MCF-7), prostate (PC3, LNCaP), lung (A549, H1473) and brain (glioblastoma, LN229), treated with the indicated concentrations of Gamitrinib (Gam) for 5 h. Mean±SEM (n=3). (b) FIG. 1b is a bar graph showing percent ATP in the various tumor cell types treated with non-mitochondrially targeted Hsp90 inhibitor, 17-AAG (20 μM), and analyzed after 5 h. Mean±SEM (n=3). (c) FIG. 1c is a bar graph showing percent ATP in the indicated normal (FF2508, MRCS) or tumor (LN229, PC3, BPH-1) cell types, incubated with 17-AAG or Gamitrinib (10 μM), and analyzed for ATP production after 5 h. Mean±SEM (n=3). FIG. 1d is a bar graph showing percent glucose consumption in LN229 cells treated with the indicated concentrations of Gamitrinib (Gam, 1, 2.5 and 5 μM), and analyzed after 5 h compared to untreated cultures (0). Mean±SEM (n=3); *, p=0.0.15-0.022. (e) FIG. 1e is a bar graph showing percent extracellular lactate content in LN229 cells treated with the indicated concentrations of Gamitrinib (1, 2.5 and 5 μM), and analyzed after 5 h compared to untreated cultures (0). Mean±SEM (n=3); *, p=0.15-0.022. (f) FIG. 1f is a line graph showing O₂ consumption in LN229 cells plated at increasing number, treated with vehicle, Gamitrinib or 17-AAG. O₂ consumption was analyzed by a fluorimetric assay. Mean±SEM (n=3), *, p=0.019; **, p=0.001. (g) FIG. 1g are bar graphs showing PC3 or LN229 cells, incubated with sodium pyruvate (Pyr) in the presence (5, 10 μM) or absence (None) of Gamitrinib and analyzed for ATP production after 7 h. Mean±SD of replicates (n=2). (h) FIG. 1h is a bar graph of fluorescence expression in LN229 cells labeled with the fluorescent dye H₂-DCFA (6 μM), treated with Gamitrinib (5-10 μM), with or without the antioxidant N-acetyl-L-cysteine (10 mM, NAC). H₂O₂ (5 mM) was used as control. Mean±SEM (n=4). (i) FIG. 1i is a bar graph showing H₂-DCFA-labeled LN229 cells treated with 10 μM Gamitrinib for the indicated time intervals and analyzed for changes in ROS production at the indicated time intervals with or without NAC. H₂O₂ was a control. Mean±SEM (n=3). (j) FIG. 1j is a bar graph and western blot of LN229 cells, transfected with control (Ctrl) or TRAP-1 directed siRNA, analyzed for changes in ATP production or extracellular lactate content. Mean±SEM (n=3); *, p=0.017; **, p=0.005.

FIGS. 2a-2h demonstrate mitochondrial Hsp90 regulation of CypD recruitment of HK-II. (a) FIG. 2a is a Western blot of LN229 cells treated with Gamitrinib (Gam) and cytosolic or mitochondrial (Mito) fractions analyzed after 5 h. COX-IV was used as a mitochondrial marker. (b) FIG. 2b is a bar graph of LN229 cells treated with 17-AAG (10 μM) or Gamitrinib, mitochondrial fractions analyzed after 5 h for hexokinase activity. Mean±SD (n=2); **, p=0.005-0.004. (c) FIG. 2c is a Western blot of LN229 cells transfected with control (Ctrl) or CypD- or TRAP-1-directed siRNA, isolated mitochondrial (Mito) or cytosol fractions analyzed after 48 h. (d) FIG. 2d is a bar graph of hexokinase activity in isolated mitochondria after 48 h in LN229 cells transfected with control (Ctrl) or CypD- or TRAP-1-directed siRNA. Mean±SD (n=2); ***, p=0.0009; **, p=0.0024. (e) FIG. 2e is a Western blot of Mitochondrial (Mito) or cytosol fractions from WT (CypD_{+/+}) or CypD_{-/-} MEFs. (f) FIG. 2f is a Western blot of CypD_{+/+}, CypD_{-/-} or CypD_{-/-} MEFs reconstituted with WT or PPIase defective H168Q mutant CypD cDNA and fractionated in cytosol or mitochondrial (Mito) extracts. (g) FIG. 2g is a western blot of LN229 cells left untreated (None) or incubated with Gamitrinib (5 μM), mixed with the indicated increasing concentrations of CHAPS, and detergent-insoluble proteins. AU, relative units. (h) FIG. 2h is a bar graph showing densitometric quantification of the protein bands of FIG. 2g.

FIGS. 3a-3i demonstrate modulation of AMPK and mTORC1 signaling by mitochondrial Hsp90s. (a-b) FIGS. 3a and 3b are western blots of LN229 cells transfected with control (Ctrl) or AMPK-directed siRNA, and total cell extracts (3a) or isolated cytosol (Cyto) or mitochondrial (Mito) (3b) fractions. (c) FIG. 3c are Western blots and bar graphs showing densitometric quantification of protein bands of tumor cell types treated with the indicated concentrations of Gamitrinib, and analyzed by Western blotting after 5 h. RU, relative units. (d) FIG. 3d is a Western blot showing Gamitrinib-treated (10 μM) LN229 cells analyzed at the indicated time. (e) FIG. 3e is a Western blot showing LN229 cells treated with metformin (5 mM) with or without Gamitrinib (5-10 μM), and analyzed after 12 h. (f) FIG. 3f is a Western blot of LN229 cells transfected with control (Ctrl) or the indicated individual siRNA sequences against TRAP-1 and isolated cytosol or mitochondrial (Mito) fractions were analyzed. (g) FIG. 3g are Western blots of the indicated tumor cell types treated with increasing concentrations of Gamitrinib and analyzed after 12 h. (h) FIG. 3h are Western blots of tumor (LN229) or normal (NIH3T3) cell types treated with Gamitrinib or 17-AAG (10 μM), and analyzed after 12 h. (i) FIG. 3i is a Western blot of LN229 cells treated with 2-DG (25 mM) and analyzed after 12 h.

FIGS. 4a-4i demonstrate tumor cytoprotection by nutrient-sensing networks. (a) FIG. 4a is a Western blot of LN229 cells treated with Gamitrinib or 17-AAG (10 μM) and analyzed after 12 h. (b-d) FIG. 4b is a Western blot of LN229 cells transfected with control (Ctrl), LKB1-directed siRNA, and analyzed after 48 h. FIG. 4c is a Western blot of LN229 cells transfected with control (Ctrl), AMPK-directed siRNA, and analyzed after 48 h. FIG. 4d is a Western blot of LN229 cells transfected with control (Ctrl), atg5-directed siRNA, and analyzed after 48 h. (e) FIG. 4e is a bar graph showing percent survival of LN229 cells treated with the inhibitor of phagosome formation, 3-MA, treated with Gamitrinib or 17-AAG (10 μM), and analyzed for cell viability by MTT. Mean±SD (n=2); **, p=0.0072 (f) FIG. 4f is a bar graph showing percent survival of LN229 cells transfected with control siRNA (Ctrl) or transfected with atg5-directed siRNA, incubated with Gamitrinib, and analyzed for cell viability by MTT. Mean±SEM (n=4). *, p=0.02. (g) FIG. 4g is a bar graph showing percent survival of LN229 cells transfected with control siRNA (Ctrl) or transfected with LKB1-directed siRNA, incubated with 17-AAG or Gamitrinib, and analyzed for cell viability by MTT. Mean±SEM (n=4). ***, p<0.0004. (h) FIG. 4h is a line graph showing LN229 cells transfected with control (squares) or HK-II (circles)-directed siRNA, treated with increasing concentrations of 17-AAG (black) or Gamitrinib (grey) and analyzed for cell viability by MTT. Mean±SD (n=2); *, p=0.02. (i) FIG. 4i is a two parameter histogram dot plot of LN229 cells transfected with control (Ctrl) or HK-II-directed siRNA, treated with vehicle (None) or Gamitrinib and analyzed for Annexin V (x-axis) and propidium iodide (PI) (y-axis) staining by multiparametric flow cytometry. The percentage of cells in each quadrant is indicated.

FIGS. 5a-5j demonstrate mitochondrial Hsp90 regulation of endoplasmic reticulum unfolded protein response (ER UPR). (a) FIG. 5a is a Western blot of PC3 cells incubated with Gamitrinib (5 μM) and analyzed at the indicated time intervals. (b) FIG. 5b is a digital image of an agarose gel. Gamitrinib-treated tumor cells were harvested at the indicated time intervals, and total RNA was amplified with primers to detect spliced (s) versus unspliced (u) Xbp1 transcripts. GAPDH was used as a control. (c) FIG. 5c is a Western blot of LNCaP cells treated with Gamitrinib, and analyzed at the indicated time intervals. (d) FIG. 5d is a bar graph showing mRNA expression of Gamitrinib-treated LNCaP cells which were harvested at the indicated time intervals, and analyzed for changes in CHOP, C/EBPβ or Grp78 mRNA expression, by quantitative PCR. Mean±SEM of replicates of a representative experiment (n=3). (e) FIG. 5e is a schematic diagram of ER stress luciferase-promoter reporter constructs used in this study. (f) FIG. 5f are bar graphs showing luciferase expression in PC3 cells transfected with the indicated luciferase-promoter reporter constructs, or a CHOP minimal promoter fused to luciferase, incubated with Gamitrinib (5 μM), tunicamycin (Tun, 2.5 μg/ml) or 2-deoxyglucose (2-DG) (25 mM) and analyzed for changes in luciferase expression after 20 h in a luminometer. Mean±SEM (n=4). None, untreated. (g) FIG. 5g are Western blots of PC3 or LN229 cells treated in the presence (+) or absence (−) of the mitochondrial uncoupler, CCCP, and analyzed after 6 or 16 h. (h) FIG. 5h are Western blots of the indicated tumor cell types incubated without (None) or with 5 μM Gamitrinib in the presence or absence of the indicated concentrations of NAC (20 or 50 μM), and analyzed after 6 h. (i) FIG. 5(i) is a Western blot of LN229 cells cultivated in the presence of the indicated glucose concentrations with or without (None) Gamitrinib (5 μM). (j) FIG. 5j are Western blots of LN229 cells treated with the indicated concentrations of Gamitrinib in the presence (+) or absence (−) of sodium pyruvate (Pyr), and analyzed after 7 h.

FIGS. 6a-6j demonstrate functional requirements of ER UPR induced by mitochondrial proteotoxic stress. (a-e) FIGS. 6a-6e are Western blots showing the indicated tumor cell types transfected with control siRNA (Ctrl), or siRNA directed to HK-II (a), AMPK (b), LKB1 (c), Grp78 (d), or the ER stress sensors IRE-1 or PERK, alone or in combination (e), incubated in the presence or absence (None) of Gamitrinib (5 μM) and analyzed 24-48 after siRNA transfection. (f) FIG. 6f are bar graphs showing densitometric quantification of normalized C/EBPβ, CHOP, Grp78, LC3-II or phosphorylated eIF2α expression bands from FIGS. 6 (a-e) under the conditions tested. Basal eIF2α levels in the absence of Gamitrinib were also calculated. (g) FIG. 6g (top portion) are Western blots of A549 or PC3 cells transfected with control siRNA (Ctrl) or the indicated individual siRNA sequences to Grp78, and analyzed after 48 h. The bottom portion are bar graphs showing siRNA-transfected cells as above analyzed for cell proliferation by direct cell counting. Mean±SEM of three independent experiments. *, p<0.05; **, p<0.01; ***, p<0.001. (h) FIG. 6h is a bar graph showing the number of cells of the indicated tumor cell types transfected with siRNA as in FIG. 6g, and analyzed for cell proliferation by direct cell counting. (i) FIG. 6i is a bar graph showing the percent viability of the indicated tumor cell types transfected with siRNA as in FIG. 6g, and analyzed by MTT. Mean±SEM (n=8, G; n=3, H); *, p=0.016; **, p=0.017-0.0055; ***, p<0.0001. (j) FIG. 6j is a bar graph showing siRNA-transfected PC3 cells as in (f) treated in the absence (None) or presence of 5 μM Gamitrinib and analyzed for cell viability by MTT. Mean±SEM of replicates of a representative experiment out of two independent determinations.

FIGS. 7a-7f demonstrate mitochondrial chaperone control of bioenergetics participates in tumor maintenance. (a) FIG. 7a is a Western blot of WT or V600E mutant BRAF melanoma cell types treated with 17-AAG (10 μM) or Gamitrinib (1, 2.5, 5 or 10 μM). (b) FIG. 7b is a Western blot of the indicated melanoma cell types incubated with Gamitrinib (10 μM). None, untreated. (c) FIG. 7c is a line graph of two WT (WM852, WM1366) and two mutant BRAF (Me1617, 451Lu) melanoma cells treated with Gamitrinib, and analyzed for cell viability by MTT. Mean±SD (n=2). (d) FIG. 7d is a Western blot of WM852 BRAF WT melanoma cells transfected with control (Ctrl) or AMPK directed siRNA. (e) FIG. 7e is a bar graph showing percent survival of WM852 melanoma cells transfected as in (d) and analyzed for Gamitrinib-mediated cell killing by MTT. Mean±SEM (n=3); **, p=0.002. (f) FIG. 7f are photomicrographs of melanoma cells with the indicated BRAF genotype grown as organotypic spheroids in 3D collagen-embedded matrices, incubated with the indicated concentrations of Gamitrinib, stained after 72 h with calcein-AM (live cells, light grey) and Topro-3 (dead cells, dark grey), and analyzed by confocal laser scanning microscopy. Representative images collected from one out of two independent determinations.

FIGS. 8a-8h demonstrate metabolic ER stress regulation of tumor progression, in vivo. (a) FIG. 8a are photomicrographs of prostate samples from TRAMP mice treated systemically with vehicle or Gamitrinib were analyzed by immunohistochemistry with antibodies to phosphorylated AMPK (p-AMPK), GRP78 or LC3-II. The histological diagnosis per each prostate tissue section is indicated. See Example 9. Right, quantification of staining intensity per each condition. Scale bars, 50 μm. (b) FIG. 8b is a bar graph showing IHC score of Grp78 in a universal tumor microarray as quantified by an immunohistochemistry (IHC) score. Each bar corresponds to the indicated tumor site. CNS, central nervous system. (c) FIG. 8c are photomicrographs of immunohistochemical reactivity of Grp78 expression in representative non-small cell lung cancer-tissue microarray (NSCLC-TMA). The two bottom images show areas of normal lung parenchyma negative for Grp78 expression (arrows) adjacent to Grp78-positive lung cancer. Magnification, ×50, bottom left image, ×100. (d) FIG. 8d is a bar graph showing a summary of Grp78 expression in NSCLC or normal lung examined in this study. The number of cases per histologic condition is indicated. In this series, thirteen cases (6%) were not evaluable, and 17 cases (8%) were negative for Grp78 expression. AdCa, adenocarcinoma; SCC, squamous cell carcinoma. IHC, immunohistochemistry. The statistical analysis for Grp78 expression in the various cohorts (t test) is as follows: NSCLC versus normal, p=1.37×10₋₃₂; AdCa versus normal, p=1.49×10-21; SCC versus normal, p=3.9×10₋₁₃; AdCa versus SCC, p=0.051. (e) FIG. 8e is a line graph showing percent survival in cases of lung adenocarcinoma (AdCa) with absent (negative) or high (positive) expression of Grp78, and analyzed for overall survival by a Kaplan-Meier curve. (f) FIG. 8f is a Western blot showing the indicated lung adenocarcinoma cell types were transfected with control (Ctrl) or Grp78-directed siRNA. (g) FIG. 8g is a bar graph showing percent viability of the indicated lung cancer cell lines transfected as in FIG. 8f, and analyzed by MTT (h) FIG. 8h is a bar graph showing cell number of the indicated lung cancer cell lines transfected as in FIG. 8f and analyzed by direct cell counting. Mean±SEM (n=6). *, p=0.035; ***, p=0.0001-0.0002.

FIG. 9 is a table showing the characteristics of the NSCLC patient series analyzed in this study.

FIGS. 10a-10d demonstrate Gamitrinib induction of AMPK signaling. (a) FIG. 10a, top portion, is a Western blot showing LN229 cells were with the indicated concentrations of Gamitrinib or 17-AAG. The bottom portion is a bar graph showing densitometric quantification of protein bands. RU, relative units. (b) FIG. 10b is a Western blot showing LN229 cells transfected with control siRNA (Ctrl) or multiple, independent siRNA sequences to AMPK, treated with Gamitrinib (Gam, 5 μM) or left untreated (None) and analyzed after 24 h. (c) FIG. 10c is a Western blot showing LN229 cells transfected with control siRNA (Ctrl) or multiple, independent siRNA sequences to LKB1, treated with Gamitrinib (Gam, 5 μM) or left untreated (None) and analyzed after 24 h. (d) FIG. 10d is a Western blot showing primary human fibroblasts FF2508 or MRCS transfected with control (Ctrl) or TRAP-1-directed siRNA. LN229 glioblastoma cells were used as control.

FIGS. 11a-11d demonstrate the regulation of mTORC1 activation by mitochondrial Hsp90s. (a) FIG. 11a is a bar graph showing the indicated tumor (PC3, LN229) or normal (NIH3T3) cell types treated with vehicle, 17-AAG or Gamitrinib (10 μM), and analyzed for ATP production after 5 h. Mean±SEM (n=3); **, p=0.006. (b) FIG. 11b is a Western blot showing LN229 cells transfected with control (Ctrl) or siRNA directed to LKB1, treated in the presence or absence (None) of Gamitrinib (5 μM). (c) FIG. 11c is a Western blot showing LN229 cells transfected with control (Ctrl) or siRNA directed to AMPK, treated in the presence or absence (None) of Gamitrinib (5 μM). (d) FIG. 11d is a Western blot showing LN229 cells transfected with control (Ctrl) or siRNA directed to HK-II treated in the presence or absence (None) of Gamitrinib (5 μM).

FIGS. 12a and 12b demonstrate induction of autophagy by Gamitrinib. (a) FIG. 12a is a photomicrograph of LN229 cells transfected with control plasmid CFP, CFP-LC3, mixed with control siRNA (Ctrl) or siRNA directed to LKB1, AMPK or atg5 and analyzed by fluorescence microscopy. Magnification, ×200. (b) FIG. 12b is a bar graph showing autophagosome formation per cell from three independent experiments, as determined by punctate LC3 fluorescence localization. A minimum of 50 CFP-LC3-positive cells assessed from at least ten random fields per sample were counted in triplicate. Mean±SEM.

FIGS. 13a-13d demonstrate Gamitrinib induction of ER UPR. (a) FIG. 13a is a Western blot showing LN229 cells treated for the indicated time intervals with 5 μM of Gamitrinib. (b) FIG. 13b is a Western blot showing LN229 cells treated for the indicated time intervals with increasing concentrations of Gamitrinib. (c) FIG. 13c are Western blots showing the indicated tumor cell types treated with the indicated increasing concentrations of Gamitrinib (Gam). (d) FIG. 13d is a Western blot showing the indicated tumor cell types treated with the ER stressor tunicamycin (0, 1, 2 μM).

FIGS. 14a-14e demonstrate tumor bioenergetics regulation of ER UPR. (a) FIG. 14a are Western blots showing the indicated tumor cell lines incubated with (+) or without (−) of the mitochondrial uncoupler, CCCP and analyzed at the indicated time intervals. (b) FIG. 14b is a Western blot showing LN229 cells treated with 2-deoxyglucose (2-DG) at the indicated increasing concentrations. (c) FIG. 14c is a Western blot showing LN229 cells treated with 2-deoxyglucose (2-DG) for the various time intervals at 25 mM. (d) FIG. 14d is a Western blot showing LN229 cells incubated with Gamitrinib (5 μM) plus 2-DG (25 mM) and analyzed after the indicated time intervals. (e) FIG. 14e is a bar graph showing percent survival of LN229 cells treated with the indicated combinations and analyzed for cell viability by MTT. Mean±SEM (n=7, 10). ***, p<0.0001.

FIG. 15 demonstrates siRNA targeting of Grp78. The indicated tumor cell lines were transfected with control (Ctrl) or Grp78-directed siRNA and analyzed by Western blotting after 24 h.

FIGS. 16a-16d demonstrate modulation of AMPK activation in melanoma cell types. (a) FIG. 16a is a Western blot showing the indicated melanoma cell types incubated with Gamitrinib (1, 2.5, 5 or 10 μM) or 17-AAG (10 μM). (b) FIG. 16b is a Western blot showing WM1366 melanoma cells transfected with control (Ctrl) or AMPK-directed siRNA. (c) FIG. 16c is a bar graph showing percent survival of WM1366 melanoma cells transfected as indicated in (b), incubated with vehicle or Gamitrinib and analyzed for cell viability by MTT. Mean±SEM (n=3). *, p=0.029. (d) FIG. 16d is a Western blot of the indicated melanoma cell types incubated with the pharmacologic inhibitor of MEK, U0126 with or without Gamitrinib.

FIGS. 17a-17c demonstrate Grp78 expression in human cancer. (a) FIG. 17a are photomicrographs of a universal tissue microarray constructed from the indicated primary tumor specimens of different origin was stained for Grp78 expression, by immunohistochemistry. Magnification, ×50 (circles), ×200 (squares). CNS, central nervous system. (b) FIG. 17b is a bar graph showing an IHC score for Grp78 expression in patients with NSCLC (AdCa) quantified according to tumor size (T1, T2, T3, T4) (c) FIG. 17c is a bar graph showing an IHC score for Grp78 expression in patients with NSCLC (AdCa) quantified according to the presence or absence of metastatic localization to lymph nodes.

FIGS. 18a and 18b demonstrate experimental conditions for Gamitrinib evaluation of tumor bioenergetics. (a) FIG. 18a is a bar graph showing percent viability of aliquots of the human tumor cell lines PC3, LNCaP, LN229, A549, or MCF-7 incubated with the indicated increasing concentrations of Gamitrinib for 5 hr and analyzed for changes in cell viability by direct cell counting. Mean±SD (n=2). (b) FIG. 18b are two parameter histogram dot plots of human glioblastoma LN229 cells left untreated (None) or incubated with 5 μM 17-AAG or Gamitrinib (Gam) for 5 hr and analyzed for changes in mitochondrial membrane potential by JC-1 staining and multiparametric flow cytometry. The mitochondrial uncoupler carbonyl cyanide 3-chlorophenylhydrazone (CCCP) was used as a control. The percentage of cells in each quadrant is indicated. FL1, green fluorescence; FL2, red fluorescence.

### DETAILED DESCRIPTION OF THE INVENTION

The methods provided herein are based on the inventors' unexpected discovery that mitochondrial Hsp90s are regulators of tumor bioenergetics, and tractable targets for cancer therapy. This discovery has led to methods and compositions useful in therapeutic treatment of patients as provided below.

### I. DEFINITIONS

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The terms “a” or “an” refers to one or more, for example, “a Gamitrinib” is understood to represent one or more Gamitrinib compounds. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

“Patient” or “subject” as used herein means a mammalian animal, including a human, a veterinary or farm animal, a domestic animal or pet, and animals normally used for clinical research. In one embodiment, the subject of these methods and compositions is a human.

The term “cancer” or “proliferative disease” as used herein means any disease, condition, trait, genotype or phenotype characterized by unregulated cell growth or replication as is known in the art. A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant. The methods described herein are useful for the treatment of cancer and tumor cells, i.e., both malignant and benign tumors, so long as the cells to be treated have mitochondrial localization of the chaperones as described herein. In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, leukemia, myeloma, lymphoma, glioma, Non-Hodgkin's lymphoma, leukemia, multiple myeloma and multidrug resistant cancer.

As used herein, the term “any intervening amount”, when referring to a range includes any number included within the range of values, including the endpoints.

### II. METHODS

One aspect of the invention provides a method of stimulating anti-tumor activity in a subject with cancer. This method is based on the inventor's discovery that inhibition of Heat Shock protein 90 (Hsp90) expressed in the mitochondria of tumor cells interferes with energy production of those cells.

In one embodiment, the invention provides a method of administering to a subject in need thereof a low dosage of a composition comprising a molecule that inhibits Hsp90 linked to a mitochondria-penetrating moiety and administering to the subject an effective amount of an inhibitor of autophagy or glycolysis. This therapeutic method prolongs the survival of cancer patients.

In another aspect, the invention provides a method of selectively inhibiting glucose consumption by a tumor cell, the method comprising administering a low dosage of an inhibitor of Hsp90. By the term “inhibiting glucose consumption”, as used herein, it is meant impairing or decreasing glucose metabolism by interfering with any step in the glycolytic pathway.

The molecular chaperone Hsp90 is a cancer nodal protein. Hsp90 accumulates in the mitochondria of cancer cells, but not normal cells. In concert with other chaperones, Hsp90 oversees fundamental mechanisms of protein folding quality control via sequential ATPase cycles. A distinctive feature of this pathway is its compartmentalization in multiple, semiautonomous, subcellular networks. Accordingly, Hsp90-directed folding controls the stability of hosts of client proteins in the cytosol, disassembles transcriptional complexes in the nucleus, regulates the early secretory pathway in the endoplasmic reticulum, and mediates cell motility in the extracellular milieu. Hsp90 and its related chaperone, Tumor Necrosis Factor Receptor-Associated Protein (TRAP-1), also bind components of a permeability transition pore in the mitochondrion, notably cyclophilin D (CypD), and antagonize its opening, preserving organelle integrity and suppressing the initiation of cell death. See, e.g., Kang et al, Journal Clinical Investigation, 119(3):454-464 (March 2009). Hsp90 and its related chaperone TRAP-1, are abundantly expressed in mitochondria of tumor, but not most normal cells, maintaining organelle proteostasis, and antagonizing mitochondrial permeability transition mediated by the matrix immunophilin, cyclophilin D (CypD).

Tumors successfully adapt to constantly changing intra- and extra-cellular environments, but the wirings of this processare still largely elusive. Adding complexity to chaperone-directed proteostasis is the role of organelle Hsp90s compartmentalized in the endoplasmic reticulum (ER), and mitochondria. The inventors' have found, as demonstrated in the Examples below, that Hsp90 chaperones compartmentalized in mitochondria, but not cytosol, are required to maintain energy production selectively in tumor cells. Interference with this process activates a global nutrient-sensing signaling network centered on phosphorylation of AMP-activated kinase (AMPK), inhibition of rapamycin-sensitive mTOR complex 1 (mTORC1), induction of autophagy, and expression of an endoplasmic reticulum (ER) unfolded protein response (UPR). This network provides non-overlapping survival signals for tumor cells, is exploited by genetically disparate cancers, and correlates with worst outcome in cancer patients.

As used herein, the term “Heat Shock Protein 90” (Hsp90) refers to Hsp90 itself, and includes any other protein which interacts with Hsp90, including, without limitation, other structurally related molecular chaperones that are overexpressed in the mitochondria of cancer cells, e.g., TRAP-1; Heat Shock 60 kDa Protein 1 (Hsp60/HspD1); and Heat shock 70 kDa protein 9 (HSPA9/mortalin).

A molecule that inhibits Hsp90 includes any molecule that specifically blocks the action of Hsp90 or interferes with the activity of Hsp90. In one embodiment, the molecule that inhibits Hsp90 is a direct inhibitor of the ATPase pocket function of Hsp90. In another embodiment, the molecule that inhibits Hsp90 is an allosteric inhibitor that binds to one side of the molecule and inhibits its global function.

In one embodiment, the molecule that inhibits Hsp90 linked to a mitochondria-penetrating moiety is Gamitrinib. As used herein, the term “Gamitrinib” refers to any one of a class of geldanamycin (GA)-derived mitochondrial matrix inhibitors. Gamintrinibs contain a benzoquinone ansamycin backbone derived from the Hsp90 inhibitor 17-(allylamino)-17-demethoxygeldanamycin (17-AAG), a linker region on the C17 position, and a mitochondrial targeting moiety, either provided by 1 to 4 tandem repeats of cyclic guanidinium (for example, a tetraguanidinium (G4), triguanidinium (G3), diguanidinium (G2), monoguanidinium (G1),) or triphenylphosphonium moiety (Gamitrinib-TPP-OH). For example, Gamitrinib-G4 refers to a Gamitrinib in which a tetraguanidinium moiety is present. For example, Gamitrinib-TPP refers to a Gamitrinib in which a triphenylphosphonium moiety is present. Also throughout this application, the use of the plural form “Gamitrinibs” indicates one or more of the following: Gamitrinib-G4, Gamitrinib-G3, Gamitrinib-G2, Gamitrinib-G1, and Gamitrinib-TPP or Gamitrinib-TPP-OH. Gamitrinib is a small molecule inhibitor of Hsp90 and TRAP-1 ATPase activity, engineered to selectively accumulate in mitochondria. In a preferred embodiment, the Gamintrinib is Gamitrinib-TPP-OH. See, e.g., United States Patent Publication No. 2009/0099080, which is hereby incorporated by reference in its entirety.

The terms “mitochondria-penetrating moiety” and “mitochondria-targeting moiety” are used herein interchangeably. In one embodiment, by “mitochondria-penetrating moiety” or “mitochondria-targeting moiety” it is meant a molecule that targets to and, together with its cargo, accumulates in mitochondria due to its: i) high affinity binding to one or more of intra-mitochondrial sites, ii) hydrophobicity and positive charge, iii) ability to enter mitochondria via carrier proteins unique to the organelle, and iv) specific metabolism by mitochondrial enzymes. In another embodiment, by “mitochondria-penetrating moiety” or “mitochondria-targeting moiety” it is meant a molecule which utilizes “electrophoresis” of the vehicle and cargo into mitochondria at the expense of negative inside membrane potential. See, e.g., Belikova et al, FEBS Lett. 2009 June 18; 583(12): 1945-1950 and United States Patent Publication No. 2009/0099080.

As used herein, a “low dosage” as it refers to the Hsp90 inhibitor, in one embodiment refers to a dosage of about 10 uM or less. In another embodiment, the low dosage is 0.5 μM. In another embodiment, the low dosage is 1 μM. In another embodiment, the low dosage is 2.5 μM. In another embodiment, the low dosage is 5 μM. In another embodiment, the low dosage is 10 μM. In another embodiment, the low dosage is 2 mg/kg of patient weight. In another embodiment, the low dosage is 2.5 mg/kg of patient weight. In another embodiment, the low dosage is 3 mg/kg of patient weight. In one embodiment, the low dosage is any intervening amount between 0.5 and 10 μM. In another embodiment, the low dosage is any intervening amount between 2 mg/kg and 3 mg/kg patient weight.

In another embodiment, the low dosage refers to a non-cytotoxic amount. As used herein, a “non-cytotoxic amount” as is refers to the Hsp90 inhibitor refers to a concentration which is insufficient to kill the targeted cell, i.e., the cancer cell. At non-cytotoxic concentrations, Gamitrinib does not trigger mitochondrial permeability transition, but produces non-lethal proteotoxic stress in mitochondria, characterized by organelle accumulation of misfolded and insoluble proteins. Mitochondrial proteotoxic stress imposed by Gamitrinib results in concentration-dependent release of hexokinase-II (HKII) from mitochondria, with its concomitant accumulation in the cytosol, resulting in a decrease in hexokinase activity. As demonstrated by the examples below, tumor cells exposed to Gamitrinib, but not 17-AAG, exhibited concentration dependent phosphorylation of AMP-activated kinase (AMPK), which in turn inhibited the rapamycin-sensitive mammalian target of rapamycin complex-1 (mTORC1), with loss of phosphorylation of mTOR, 4EBP1 and p70S6. Thus, in one embodiment, a non-cytotoxic amount is a concentration sufficient to produce the above-described effects in tumor cells. The dosage required for a non-cytotoxic amount will depend primarily on factors such as the condition being treated, the age, weight and health of the patient, and may thus vary among patients. In one embodiment, a non-cytotoxic amount is amount is less than 10 μM Gamitrinib. In another embodiment, the non-cytotoxic amount is 0.5 μM. In another embodiment, the non-cytotoxic amount is 1 μM. In another embodiment, the non-cytotoxic amount is 2.5 μM. In another embodiment, the non-cytotoxic amount is 50 μM. In another embodiment, the non-cytotoxic amount is 10 μM. In one embodiment, the non-cytotoxic amount is any intervening amount between 0.5 and 10 μM.

As used herein, an “inhibitor of autophagy” means a chemical, compound or agent which inhibits the processing and degradation of cytoplasmic components and organelles by the lysosome/vacuole. An “inhibitor of glycolysis” means a chemical, compound or agent which inhibits any step in the metabolic process by which glucose is converted to pyruvate. In one embodiment, the inhibitor of autophagy or glycolysis is 2-deoxyglucose. In another embodiment, the inhibitor of autophagy is 3-methyladenine (3-MA). In another embodiment, the inhibitor of autophagy is cholorquine or hydroxychloroquine. In another embodiment, the inhibitor of autophagy is a PI3K inhibitor. In another embodiment, the inhibitor of glycolysis is oxamate, or 3-bromopyruvate (3-BrPA).

As used herein, the term “effective amount” or “pharmaceutically effective amount” as it refers to the inhibitor of autophagy/glycolysis refers to the amount of active compound or pharmaceutical agent that elicits the biological or medicinal response in a tissue, system, animal, individual or human that is being sought by a researcher, veterinarian, medical doctor or other clinician, which includes one or more of the following, preventing a disease; e.g., inhibiting a disease, condition or disorder in an individual that is experiencing or displaying the pathology or symptomatology of the disease, condition or disorder (i.e., arresting or slowing further development of the pathology and/or symptomatology); ameliorating a disease, condition or disorder in an individual that is experiencing or displaying the pathology or symptomatology of the disease, condition or disorder (i.e., reversing the pathology and/or symptomatology); and inhibiting a physiological process. For example, an effective amount of an inhibitor of autophagy/glycolysis, when administered to a subject to treat cancer, is sufficient to inhibit, slow, reduce, or eliminate tumor growth in a subject having cancer. In one embodiment, the effective amount of the above-noted inhibitor is sufficient to inhibit autophagy or glycolysis or inhibit Hsp90 activity.

In one embodiment, the inhibitor of autophagy or glycolysis is 2-deoxyglucose. In one embodiment, the effective amount of 2-deoxyglucose is 4 mM to 25 mM. In another embodiment, the effective amount is 5 mM. In another embodiment, the effective amount is 10 mM. In another embodiment, the effective amount is 15 mM. In another embodiment, the effective amount is 20 mM. In another embodiment, the effective amount is 25 mM. In another embodiment, the effective amount is 1 mg/kg to 1 g/kg of patient weight. In another embodiment, the effective amount is 30 mg/kg of patient weight. In another embodiment, the effective amount is 5 mg/kg of patient weight. In another embodiment, the effective amount is 10 mg/kg of patient weight. In another embodiment, the effective amount is 15 mg/kg of patient weight. In another embodiment, the effective amount is 20 mg/kg of patient weight. In another embodiment, the effective amount is 25 mg/kg of patient weight. In another embodiment, the effective amount is 35 mg/kg of patient weight. In another embodiment, the effective amount is 40 mg/kg of patient weight. In another embodiment, the effective amount is 45 mg/kg of patient weight. In another embodiment, the effective amount is 50 mg/kg of patient weight. In another embodiment, the effective amount is 60 mg/kg of patient weight. In another embodiment, the effective amount is 75 mg/kg of patient weight. In another embodiment, the effective amount of the inhibitor of autophagy/glycolysis is 100 mg/kg of patient weight.

In one embodiment, the inhibitor of autophagy or glycolysis is 3-methyladenine. In one embodiment, the effective amount of 3-MA is 1 mM to 25 mM. In another embodiment, the effective amount is 5 mM. In another embodiment, the effective amount is 10 mM.

In another embodiment, the inhibitor of autophagy or glycolysis is hydroxychloroquine. In one embodiment, the effective amount of hydroxychloroquine ranges between 400-600 mg/day, 200-400 mg/day, or 400-800 mg/day. In one embodiment, the effective amount is 200 mg/day. In one embodiment, the effective amount is 300 mg/day. In one embodiment, the effective amount is 400 mg/day. In one embodiment, the effective amount is 500 mg/day. In one embodiment, the effective amount is 600 mg/day. In one embodiment, the effective amount is 700 mg/day. In one embodiment, the effective amount is 800 mg/day.

In another embodiment, the inhibitor of autophagy or glycolysis is 3-BrPA. In one embodiment, the effective amount of 3-BrPA 0.5 to 2.5 mM. In another embodiment, the effective amount is 0.5 mM. In another embodiment, the effective amount is 1.0 mM. In another embodiment, the effective amount is 1.5 mM. In another embodiment, the effective amount is 2 mM. In another embodiment, the effective amount is 2.5 mM.

The therapeutic compositions administered in the performance of these methods, e.g., a molecule that inhibits Hsp90 linked to a mitochondria-penetrating moiety and an inhibitor of autophagy or glycolysis, may be administered directly into the environment of the targeted cell undergoing unwanted proliferation, e.g., a cancer cell or targeted cell (tumor) microenvironment of the patient. In an alternative embodiment, the compositions are administered systemically, without regard to the location of the cancer, i.e., parenteral administration. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically. Dosages may be administered continuously for a certain period of time, or periodically every week, month, or quarter, dependent on the condition and response of the patient, as determined by a physician.

In one embodiment, the compositions i.e., a molecule that inhibits Hsp90 linked to a mitochondria-penetrating moiety and an inhibitor of autophagy or glycolysis, are administered at the same time. In another embodiment, the compositions are administered sequentially. In another embodiment, the molecule that inhibits Hsp90 is administered first. In another embodiment, the inhibitor of autophagy or glycolysis is administered first. In another embodiment, the compositions are administered within a suitable period, e.g., hours, days or weeks of each other. These periods may be determined by a physician.

In one embodiment, the compositions are administered periodically, e.g. every day, week, two weeks, monthly, quarterly, or as prescribed by physician.

These therapeutic compositions may be administered to a patient preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

In one embodiment, the methods described herein include administration of the molecule that inhibits Hsp90 linked to a mitochondria-penetrating moiety and an inhibitor of autophagy or glycolysis, as described above, in combination with other known anti-proliferative disease therapies. In one embodiment of such combination therapy, the present method can include administration of a passive therapeutic that can immediately start eliminating the targeted cell undergoing unrestricted or abnormal replication or proliferation, e.g., tumor. This can also be accompanied by administration of active immunotherapy to induce an active endogenous response to continue the tumor eradication. Such immune-based therapies can eradicate residual disease and activate endogenous antitumor responses that persist in the memory compartment to prevent metastatic lesions and to control recurrences. This treatment may occur, before, during or after administration of the molecule that inhibits Hsp90 linked to a mitochondria-penetrating moiety and an inhibitor of autophagy or glycolysis. In another example, surgical debulking, in certain embodiments is a necessary procedure for the removal of large benign or malignant masses, and can be employed before, during or after application of the methods and compositions as described herein. Chemotherapy and radiation therapy, in other embodiments, bolster the effects of the methods described herein. Such combination approaches (surgery plus chemotherapy/radiation plus immunotherapy) with the methods of administering Gamitrinib and an inhibitor of autophagy/glycolysis are anticipated to be successful in the treatment of many proliferative diseases.

### III. DIAGNOSTIC METHODS

In another aspect, the invention provides a method of diagnosing cancer in a subject comprising measuring the level of Glucose-regulated protein 78 (Grp78) in a sample taken from the subject and comparing the level of Grp78 to a control or standard level. This aspect is based on the inventor's discovery that, in most cancers, Grp78 expression correlates with both presence of cancer and decreased survival time in cancer subjects. The amino sequence of human Grp78 is known in the art and can be found at NCBI accession no. NP_005338; the mRNA sequence can be found at NCBI accession no. NM_005347. As exemplified in the Examples below, Grp78 is upregulated in genetically disparate cancers, particularly lung cancers.

Grp78 is a well-characterized molecular chaperone that is ubiquitously expressed in mammalian cells. Grp78 binds to hydrophobic patches on nascent polypeptides within the endoplasmic reticulum (ER) and plays a role in signaling the unfolded protein response (UPR). Grp78 is induced in tumor cells by Gamitrinib (See, Example 6 below) or nutrient deprivation.

In one embodiment, a method is provided for diagnosing a proliferative disease or disorder, e.g., cancer, in a subject. The method includes measuring the level of expression or activity of Grp78 in a biological sample from a mammalian subject, preferably a human subject. When compared to the level of expression or activity of Grp78 in a healthy mammalian subject, an increased level of expression or activity is an indication of a diagnosis of proliferative disease or disorder. In one embodiment, the presence of a detectable level of Grp78 is indicative of proliferative disease or disorder.

In another aspect, a diagnostic method for proliferative disease or disorder involves measuring the level of expression or activity of Grp78 and at least one additional biomarker in the above-noted biological sample. The combined changes in expression or activity of Grp78 and the additional biomarker from their respective levels of expression or activity in a healthy mammalian subject is an indication or differential indication of a diagnosis of proliferative disease or disorder.

In another aspect, a method is provided for monitoring progression of proliferative disease or disorder in a mammalian subject suffering from that disorder. In this method the level of expression or activity of Grp78 in a biological sample from a mammalian subject having proliferative disease or disorder is measured and compared to the level of expression or activity of Grp78 of a temporally earlier biological sample of the same subject. In this method, a decreased expression or activity level of the Grp78, compared to that in an earlier biological sample of the same subject is indicative of regression or improvement in the disorder. Conversely, an increase in Grp78 in the later sample is indicative of progression or increase in severity of the disorder. As one embodiment, this method can be applied to a subject being treated for proliferative disease, e.g., cancer. In this circumstance, the method enables a determination of the efficacy of the treatment.

Conventional diagnostic assay methods for detection of protein, nucleic acid, or enzymatic activity may be selected by one of skill in the art given the teachings provided herein. For example, in one embodiment, a method of evaluating the probability of the presence of malignant cells can be performed in a group of cells freshly removed from a host. Such methods can be used to detect abnormal cell growth or replication, e.g., tumors, quantitate and monitor their growth, and help in the diagnosis and prognosis of proliferative disease. For example, an increase in the level or activity of Grp78 from that of a standard is indicative of the presence of the proliferative disease, e.g., cancer. In one embodiment, the presence of a detectable level of Grp78 is indicative of the presence of cancer.

Such methods involve comparing the amount of Grp78 quantitated in a sample from a subject being tested to a predetermined standard or cut-off value. A standard may correspond to levels quantitated for another sample or an earlier sample from the subject, or levels quantitated for a control sample, in particular a sample from a subject with a lower grade cancer, or a healthy subject. Levels for control samples from healthy subjects or cancer subjects may be established by prospective and/or retrospective statistical studies. Healthy subjects who have no clinically evident disease or abnormalities may be selected for statistical studies. Diagnosis may be made by a finding of statistically different levels of Grp78 compared to a control sample or previous levels quantitated for the same subject.

In other embodiments, the diagnostic methods use multiple markers for a proliferative disease. In one embodiment, a method analyses a biological sample for the presence of Grp78 and other markers that are specific indicators of the proliferative disease or cancer. The methods described herein may be modified by including reagents to detect the markers or polynucleotides encoding the markers.

In one embodiment, the biomarker or its activity may be detected based on the level of a polynucleotide encoding Grp78 (or fragment thereof) in a sample. Techniques for detecting nucleic acid molecules such as polymerase chain reaction (PCR) and hybridization assays are well known in the art. Probes may be used in hybridization techniques to detect polynucleotides. The technique generally involves contacting and incubating nucleic acids obtained from a sample from a patient or other cellular source with a probe under conditions favorable for the specific annealing of the probes to complementary sequences in the nucleic acids (e.g. under stringent conditions as discussed herein). After incubation, the non-annealed nucleic acids are removed, and the presence of nucleic acids that have hybridized to the probe, if any, are detected. Nucleotide probes for use in the detection of polynucleotide sequences in samples may be constructed using conventional methods known in the art. The probes may comprise DNA or DNA mimics corresponding to a portion of an organism's genome, or complementary RNA or RNA mimics. The nucleic acids can be modified at the base moiety, at the sugar moiety, or at the phosphate backbone. DNA can be obtained using standard methods such as polymerase chain reaction (PCR) amplification of genomic DNA or cloned sequences. Computer programs known in the art can be used to design primers with the required specificity and optimal amplification properties.

A nucleotide probe may be labeled with a detectable substance such as a radioactive label which provides for an adequate signal and has sufficient half-life such as ³²P, ³H, ¹⁴C or the like. Other detectable substances that may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleic acids to be detected and the amount of nucleic acids available for hybridization. Labeled probes may be hybridized to nucleic acids on solid supports such as nitrocellulose filters or nylon membranes as generally described in Sambrook et al., 1989, Molecular Cloning, A Laboratory Manual (2nd ed.). The nucleic acid probes may be used to detect Grp78, preferably in human cells. The nucleotide probes may also be useful in the diagnosis of cancer, involving Grp78 in monitoring the progression of cancer, or monitoring a therapeutic treatment.

The detection of polynucleotides in a sample may involve the amplification of specific gene sequences using an amplification method such as PCR, followed by the analysis of the amplified molecules using techniques known to those skilled in the art. By way of example, oligonucleotide primers may be employed in a PCR based assay to amplify a portion of a polynucleotide and to amplify a portion of a polynucleotide derived from a sample, wherein the oligonucleotide primers are specific for (i.e. hybridize to) the polynucleotides. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of Grp78; that is, they are at least 10 nucleotides, and preferably at least 20 nucleotides in length. In an embodiment the primers and probes are at least about 10-40 nucleotides in length.

Hybridization and amplification reactions may also be conducted under stringent conditions as discussed herein. Hybridization and amplification techniques described herein may be used to assay qualitative and quantitative aspects of polynucleotide expression. For example, RNA may be isolated from a cell type or tissue known to express Grp78, and tested utilizing the hybridization (e.g. standard Northern analyses) or PCR techniques.

In another aspect, a method is provided employing reverse transcriptase-polymerase chain reaction (RT-PCR), in which PCR is applied in combination with reverse transcription. Generally, RNA is extracted from a sample using standard techniques and is reverse transcribed to produce cDNA. The cDNA is used as a template for a polymerase chain reaction. The cDNA is hybridized to primer sets which are specifically designed against a Grp78. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected cancer, an subject who is not afflicted with cancer or has early stage disease or has aggressive or metastatic disease. The reaction may be performed on several dilutions of cDNA spanning at least two orders of magnitude. A statistically significant difference in expression in several dilutions of the subject sample as compared to the same dilutions of the standard, e.g., a non-cancerous sample or early-stage cancer sample may be considered positive for the presence of cancer.

In an aspect, a diagnostic method for monitoring or diagnosing proliferative disease in a subject involves quantitating a Grp78 polypeptide (or a fragment thereof) in a biological sample from the subject comprising reacting the sample with antibodies specific for Grp78 which are directly or indirectly labeled with detectable substances and detecting the detectable substances.

In an aspect of the invention, a method for detecting or diagnosing cancer is provided comprising or consisting essentially of: (a) obtaining a sample of cells suspected of expressing a level or activity of an Grp78 polypeptide; (b) contacting said sample with antibodies that specifically bind Grp78 polypeptide under conditions effective to bind the antibodies and form complexes; (c) measuring the amount of Grp78 polypeptide present in the sample by quantitating the amount of the complexes; and (d) comparing the amount of Grp78 polypeptide present in the samples with the amount of Grp78 polypeptide in a control, wherein a change or significant difference or increase in the amount or activity of Grp78 in the sample compared with the amount or activity in the control is indicative of a proliferative disease, e.g., cancer, stage of cancer, progression, aggressiveness and/or metastatic potential of the disease.

In one embodiment, the method for monitoring the progression of a proliferative disease in an subject, comprises: (a) contacting an antibody which binds to Grp78 polypeptide with a sample from the subject, so as to form a complex comprising the antibody and Grp78 in the sample; (b) determining or detecting the presence or amount of complex formation in the sample; (c) repeating steps (a) and (b) at a point later in time; and (d) comparing the result of step (b) with the result of step (c), wherein a difference in the amount of complex formation is indicative of disease, disease stage, progression, aggressiveness and/or metastatic potential of the proliferative disorder in the subject. The amount of complexes may also be compared to a value representative of the amount of the complexes from a subject not at risk of, or afflicted with cancer at a different stage.

Antibodies may be used in any immunoassay that relies on the binding interaction between antigenic determinants of Grp78 and the antibodies. Immunoassay procedures for in vitro detection of antigens in samples are also well known in the art. (See Sambrook et al, and other known publications for a general description of immunoassay procedures). Qualitative and/or quantitative determinations of Grp78 in a sample may be accomplished by competitive or non-competitive immunoassay procedures in either a direct or indirect format. Detection of Grp78 using antibodies can, for example involve immunoassays which are run in either the forward, reverse or simultaneous modes. Examples of immunoassays are radioimmunoassays (RIA), enzyme immunoassays (e.g. ELISA), immunofluorescence, immunoprecipitation, latex agglutination, hemagglutination, histochemical tests, and sandwich (immunometric) assays. Alternatively, the binding of antibodies to Grp78 can be detected directly using, for example, a surface plasmon resonance (SPR) procedure such as, for example, Biacore®, microcalorimetry or nano-cantilivers. These terms are well understood by those skilled in the art, and they will know, or can readily discern, other immunoassay formats without undue experimentation.

Antibodies specific for Grp78 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I) fluorescent labels, (e.g., FITC, rhodamine, lanthanide phosphors), luminescent labels such as luminol; and enzymatic labels (e.g., horseradish peroxidase, beta-galactosidase, luciferase, alkaline phosphatase, acetylcholinesterase), biotinyl groups (which can be detected by marked avidin e.g., streptavidin containing a fluorescent marker or enzymatic activity that can be detected by optical or calorimetric methods), and predetermined polypeptide epitopes recognized by a secondary reporter (e.g., leucine zipper pair sequences, binding sites for secondary antibodies, metal binding domains, epitope tags). In some embodiments, labels are attached via spacer arms of various lengths to reduce potential steric hindrance. Antibodies may also be coupled to electron dense substances, such as ferritin or colloidal gold, which are readily visualised by electron microscopy.

One of the ways an antibody can be detectably labelled is to link it directly to an enzyme. The enzyme when later exposed to its substrate will produce a product that can be detected. Examples of detectable substances that are enzymes are horseradish peroxidase, beta-galactosidase, luciferase, alkaline phosphatase, acetylcholinesterase, malate dehydrogenase, ribonuclease, urease, catalase, glucose-6-phosphate, staphylococcal nuclease, delta-5-steroid isomerase, yeast alcohol dehydrogenase, alpha-glycerophosphate, triose phosphate isomerase, asparaginase, glucose oxidase, and acetylcholine esterase.

For increased sensitivity in an immunoassay system a fluorescence-emitting metal atom such as Eu (europium) and other lanthanides can be used. These can be attached to the desired molecule by means of metal-chelating groups such as DTPA or EDTA. A bioluminescent compound may also be used as a detectable substance. Examples of bioluminescent detectable substances are luciferin, luciferase and aequorin. Similarly a fluorescent protein, such as GFP, BFP etc, may be used as a reporter.

Indirect methods may also be employed in which the primary antigen-antibody reaction is amplified by the introduction of a second antibody, having specificity for the antibody reactive against Grp78. By way of example, if the antibody having specificity against Grp78 is a rabbit IgG antibody, the second antibody may be goat anti-rabbit IgG, Fc fragment specific antibody labeled with a detectable substance as described herein.

Methods for conjugating or labelling the antibodies discussed above may be readily accomplished by one of ordinary skill in the art.

In the context of the methods of the invention, the sample, binding agents (e.g. antibodies), or Grp78 may be immobilized on a carrier or support, such as, for example, agarose, cellulose, nitrocellulose, dextran, Sephadex, Sepharose, liposomes, carboxymethyl cellulose, polyacrylamides, polystyrene, filter paper, ion-exchange resin, plastic film, nylon or silk. The support material may have any possible configuration including spherical, cylindrical, or flat. Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. Binding agents (e.g. antibodies) may be indirectly immobilized using second binding agents specific for the first binding agent. For example, mouse antibodies specific for Grp78 may be immobilized using sheep anti-mouse IgG Fc fragment specific antibody coated on the carrier or support.

Aspects of the methods of the invention involve (a) reacting a biological sample from a subject with an antibody specific for Grp78 wherein the antibody is directly or indirectly labelled with enzymes; (b) adding substrates for the enzymes wherein the substrates are selected so that the substrates, or reaction products of the enzymes and substrates form fluorescent complexes; (c) quantitating Grp78 polypeptide in the sample by measuring fluorescence of the fluorescent complexes; and (d) comparing the quantitated levels to levels obtained for other samples from the subject patient, or control subjects.

In another aspect, a diagnostic composition or kit for diagnosing or differentially diagnosing the occurrence, stage or progression of proliferative disease or disorder in a mammalian subject is provided. In one aspect, the composition contains one or a plurality of polynucleotides immobilized on a substrate, wherein at least one polynucleotide is a genomic probe that hybridizes to Grp78 mRNA. In another aspect, the composition contains one or a plurality of PCR primer-probe sets, wherein at least one primer-probe set amplifies a polynucleotide (mRNA) sequence of Grp78. In another aspect, the composition contains one or a plurality of ligands, such as antibodies or fragments, wherein at least one ligand binds to Grp78 in a biological sample of a mammalian subject. In other embodiments, the other polynucleotides or other primer-probe sets or other ligands are designed to detect additional biomarkers. Such diagnostic compositions may also contain conventional labels which emit detectable signals when complexes with the Grp78 in the sample are formed.

Still other known methods and conventional, as well as commercial assays and components, may be employed in the practice of these methods for screening and diagnosis.

### IV. EXAMPLES

The invention is now described with reference to the following examples. These examples are provided for the purpose of illustration only. The compositions, experimental protocols and methods disclosed and/or claimed herein can be made and executed without undue experimentation in light of the present disclosure. The protocols and methods described in the examples are not considered to be limitations on the scope of the claimed invention. Rather this specification should be construed to encompass any and all variations that become evident as a result of the teaching provided herein. One of skill in the art will understand that changes or variations can be made in the disclosed embodiments of the examples, and expected similar results can be obtained. For example, the substitution of reagents that are chemically or physiologically related for the reagents described herein are anticipated to produce the same or similar results. All such similar substitutes and modifications are apparent to those skilled in the art and fall within the scope of the invention.

### Example 1: Materials and Methods

I. Antibodies and Reagents.

The following antibodies to hexokinase-I (HK-I, Cell Signaling), hexokinase-II (HK-II, Cell Signaling), Cox-IV (Cell Signaling), LC-3 (Cell Signaling), Thr172-phosphorylated AMPKα (Cell Signaling), AMPKα (Cell Signaling), Ser2448-phosphorylated mammalian Target of Rapamycin (mTOR, Cell Signaling), Thr389-phosphorylated p70S6 kinase (Cell Signaling), Thr37/46-phosphorylated 4EBP1 (Cell Signaling), VDAC (Cell Signaling), CypD (Calbiochem), Hsp90 (BD Biosciences), TRAP-1 (BD Biosciences), IRE1α (Cell Signaling), PERK (Cell Signaling), CHOP (Cell Signaling), C/EBPβ (Cell Signaling), Grp78 (Cell Signaling), β-tubulin (Sigma-Aldrich), and β-actin (Sigma-Aldrich) were used.

The endoplasmic reticulum (ER)-stress reporters (ERSEII-FL, UPRE-FL, ATF4REFLuc) were a kind gift of Dr. Winklhofer, and characterized previously. A CHOP minimal promoter construct (nucleotides −954; +91) cloned upstream of a luciferase reporter gene (CHOP-FLuc) was the kind gift of Dr. Fafournoux, and characterized previously. The complete chemical synthesis, HPLC profile, and mass spectrometry of mitochondrial-targeted small molecule Hsp90 antagonist Gamitrinib (GA mitochondrial matrix inhibitors) has been reported previously. The Gamitrinib variant containing triphenylphosphonium as mitochondrial-targeting moiety was used in this study. Non-mitochondrially permeable Hsp90 inhibitor 17-allylamino demethoxygeldanamycin (17-AAG) was obtained from LC-Laboratories. 2-deoxy-D-glucose (2-DG), 3-methyladenine (3-MA) and tunicamycin were obtained from Sigma-Aldrich.

II. Cell Culture.

Human glioblastoma LN229, prostate adenocarcinoma PC3 or LNCaP cells, or normal NIH3T3 fibroblasts were obtained from the American Tissue Culture Collection (ATCC, Manassas, Va.), and maintained in culture according to the supplier's specifications. The following wild type (WT) BRAF N-Ras mutant melanoma cells WM852 and WM1366, and melanoma cells carrying a V600E activating mutation in the BRAF oncogene, 451Lu, Me11617 and WM983B were used in this study. WT or CypD knockout Mouse Embryonic Fibroblasts (MEFs, CypD−/−) were characterized earlier 40. In some experiments, CypD−/− MEFs were stably transfected with control plasmid, WT CypD or peptidyl prolyl isomerase (PPIase)-deficient H168Q CypD mutant cDNA, and analyzed for differential recruitment of HK-II to mitochondrial or cytosolic fractions.

III. Transfections.

Gene knockdown experiments were carried out using control, nontargeting siRNA pool (Dharmacon, cat. no. D-001810) or specific ON-Target SMARTpool siRNAs to TRAP-1 (Dharmacon, cat. no. L-010104), CypD (Dharmacon, cat. no. L-009708), HK-II (Dharmacon, cat. no. L-006735), atg5 (Dharmacon, cat. no. L-004374), LKB1 (Dharmacon, cat. no. L-005035), CHOP (Dharmacon, cat. no. L-004819-00-0005), C/EBPβ (Dharmacon, cat. no. L-006423-00-0005), and Grp78 Dharmacon, cat. no. L-008198-00-0005), AMPKα1/α2 (Santa Cruz Biotechnology, cat. no. sc-45312), IRE-1α (Santa Cruz Biotechnology, cat. no. Sc-40705) and PERK (Santa Cruz Biotechnology, cat. no. Sc-36213). Individual ON-Target SMART siRNA were used for Grp78 (Dharmacon, cat. no. J-008198-06, -07, -08 and -09).

To examine the role of ER UPR on Gamitrinib-mediated killing, PC3 or LN229 cells (5×10⁵) were reverse transfected with 10 nM siRNA to Grp78, CHOP or C/EBPβ in the presence of Lipofectamine RNAiMAX (Invitrogen), and immediately plated in duplicate at 3700 cells/well in 96-MW plates. Cell viability was estimated with a 3 (4,5-dimethyl-thyazoyl-2-yl)2,5 diphenyltetrazolium bromide (MTT) colorimetric assay, as described previously. Data were background subtracted relative to vehicle-treated cultures. Alternatively, proliferation in control cultures or GRP78-silenced cultures (2×10⁴-1.25×10⁵ cells) was analyzed after 3-4 d by direct cell counting. At least 2-3 independent experiments were performed.

In another series of experiments, PC3 cells were transfected with control or Grp78-directed siRNA, and analyzed for cell viability after 24 h by an MTT assay. For promoter analysis, PC3 cells were plated in triplicate onto 48-well plates at 1.4×10⁴ cells/well, and cotransfected with Firefly Luciferase-reporters for IRE1, ATF6, PERK or CHOP and a constitutive reporter (pTK-Renilla Luc) in a 10:1 ratio using X-tremeGENE reagent (Roche). After 24 h, cells were treated with vehicle, Gamitrinib, tunicamycin or 2-DG and analyzed after 20 h for Firefly and Renilla luciferase activity using a Dual Luciferase Reporter Assay System (Promega). Transfection of plasmid DNAs was carried out with lipofectamine 2000 (Invitrogen) according to published protocols. A PPIase-defective CypD H168Q mutant cDNA was generated by Quick-Change site-directed mutagenesis (Stratagene) using primers 5′-CTGGTTGGATGGCAAGCAGGTTGTGTTCGGTCACG-3′ (forward) and 5′-CGTGACCGAACACAACCTG CTTGCCATCCAACCAG-3′ (reverse) (mutated sequences are bolded). The inserted mutation (bolded) was confirmed by DNA sequencing, and cloned in a pcDNA3.0 expression vector (Invitrogen) for reconstitution experiments in CypD−/− MEFs.

IV. Subcellular Fractionation.

Mitochondrial fractions were isolated from Gamitrinib treated LN229 cells (0-20 μM for 5 h) using an ApoAlert™ cell fractionation kit (CLONTECH). Briefly, LN229 cells were mechanically disrupted by 60 strokes with a Dounce homogenizer in isolation buffer containing 1 mM DTT plus protease inhibitor cocktail. Cell debris was removed by centrifugation at 700 g for 10 min. The supernatant was further centrifuged at 10,000 g for 25 min, and supernantants or mitochondrial pellets were processed for further analysis.

V. Protein Analysis.

For Western blotting, protein lysates were prepared from the different cell types in RIPA Buffer (150 mM NaCl, 1.0% Triton X-100, 0.5% sodium deoxycholate, 0.1% SDS, 50 mM Tris, pH 8.0) containing EDTA-free Protease Inhibitor Cocktail (Roche) and Phosphatase Inhibitor Cocktail 2 and 3 (Sigma Aldrich). Equal amounts of protein lysates were separated by SDS gel electrophoresis, transferred to PVDF membranes and incubated with primary antibodies of various specificities. Protein bands were detected by chemiluminescence.

VI. Mitochondrial Protein Folding.

Mitochondrial protein folding assays were performed as described (Moisoi et al., 2009). Briefly, mitochondrial fractions were isolated from vehicle- or Gamitrinib-treated LN229 cells (5 μM for 12 h) and suspended in equal volume of mitochondrial fractionation buffer containing increasing concentrations of CHAPS (0, 0.05, 0.1, 0.2, 0.5, 1 or 2%). Samples were incubated for 20 min on ice and detergent-insoluble protein aggregates were isolated by centrifugation (20,000 g) for 20 min. Pellets were processed for further analysis.

VII. Glucose and Lactate Determination.

Glucose concentrations in cell culture media were determined using a Glucose kit (Sigma-Aldrich). Briefly, 2×106 cells were seeded in 10 cm2 tissue culture dishes for 48 h, mixed with DMEM in the presence of Gamitrinib or 17-AAG (0-20 μM) for additional 4 h, and 200 μl aliquots of culture medium were incubated with 1 ml assay mixture, containing 1.5 mM NAD, 1 mM ATP, 1.0 U/ml Hexokinase, and 1.0 U/ml G6PDH. The glucose concentration under the various conditions was determined by measuring the amount of reduced NAD to NADH by G6PDH, and quantified spectrophotometrically at 340 nm wavelength. Extracellular lactate was measured by a colorometric assay kit (Abcam). Culture media from LN229 cells was replaced with DMEM containing vehicle (DMSO) or Gamitrinib (5 μM) for 5 h. Changes in lactate concentrations were measured by analysis of lactate-dependent conversion of NADP to NADPH in the presence of excess lactate dehydrogenase (LDH), and quantified by absorbance at 450 nm. All assays were performed at 25° C. under conditions of linear lactate-limited NADPH formation.

VIII. Oxygen Consumption.

Treated tumor cells were analyzed using a fluorescence oxygen sensitive probe-based oxygen measuring kit (Luxcel Bioscience). For these experiments, LN229 cells were plated at increasing cell density (10-60×10⁴/ml) on black body, clear bottom 96-well plates. The culture medium was replaced with 150 μl of phenol free DMEM containing 10% fetal bovine serum in the presence of 17-AAG or Gamitrinib (10 μM). Cells were further incubated with an oxygen-sensing probe (10 pmol/well), and 100 μl of heavy mineral oil was added to each well to seal the samples from ambient oxygen. After a 2 h incubation at 37° C., oxygen consumption was determined by quantifying the probe fluorescence signal in each well using a plate reader (Beckman Coulter) with excitation and emission wavelengths at 370 nm and 625 nm, respectively.

IX. ATP Measurement.

Intracellular ATP concentrations were measured by the luciferin-luciferase method using an ATP measuring kit (Biochain). The ATP concentration in each extract was measured with a microplate luminometer (Beckman Coulter) against standard ATP solutions used as reference. In some experiments, PC3 or LN229 cells were preincubated with sodium pyruvate for 7 h in the presence of vehicle or Gamitrinib before determination of ATP production.

X. HK Activity.

HK activity in LN229 cells was measured as the total glucosephosphorylating activity using a standard G6PDH-coupled assay kit (BioVision). Mitochondria isolated from Gamitrinib- or 17-AAG-treated LN229 cells, or cultures transfected with various siRNAs were homogenized in cold PBS, followed by centrifugation at 1000 g for 10 min at 4° C. HK activity was determined by analysis of G6P-dependent conversion of NADP to NADPH in the presence of excess G6PDH in the presence of 2 mM glucose, and quantification of absorbance at 450 nm. All assays were performed at 25° C. under conditions of linear HK-limited NADPH formation.

XI. Genetic Model of Prostate Cancer.

All experiments involving vertebrate animals were approved by an Institutional Animal Care and Use Committee at the University of Massachusetts Medical School and The Wistar Institute. Activation of mitochondrial HSP90 signaling was investigated in immunocompetent TRAMP (Transgenic Adenocarcinoma of the Mouse Prostate) mice, as described (Kang et al., Targeted inhibition of mitochondrial HSP90 suppresses localised and metastatic prostate cancer growth in a genetic mouse model of disease. Br J Cancer 104, 629-634.

XII. Xbp1 Splicing Assay.

Total RNA was extracted from Gamitrinib-treated prostate cancer cells with TriReagent (Ambion), treated with DNaseI (Fermentas) for 30 min at 37° C. and retro-transcribed to cDNA using the Maxima Universal First Strand cDNA Synthesis kit (Fermentas). One μl of each RT reaction was amplified by PCR using Xpb1-directed primer pairs, TTACGAGAGAAAACTCATGGCC (forward) and GGGTCCAAGTTGTCCAGAATGC (reverse). GAPDH amplification was carried out with primer pairs, CAAGGTCATCCATGACAACTTTG (forward) and GTCCACCACCCTGTTGCTGTAG (reverse). Reaction conditions were as follows: 95° C. for 15 min, 94° C. for 30 sec, 61° C. for 30 sec, 72° C. for 30 sec for 30 cycles, and extension at 72° C. for 10 min. PCR products were separated on 2% Metaphor Agarose (Lonza) in TBE buffer, and visualized in the presence of 0.4 μg/ml Ethidium Bromide.

XIII. RT-qPCR.

Total RNA from Gamitrinib-treated tumor cells was extracted and retro-transcribed to cDNA as described above. qPCR reactions were carried out in triplicates using an ABI7500 Fast Real Time PCR instrument with 1 μl of a 1:20 dilution of RT product, Maxima SYBR Green qPCR Master Mix (Fermentas) plus the addition of ROX 10 nM as passive reference dye, in a total volume of 25 μl. The following primer pairs were used: CHOP (forward): TGTTAAAGATGAGCGGGTGG; CHOP (reverse): AGGTGTGGTGATGTATGAAGAT; C/EBPβ (forward): TTGTTTTGTTTTTTGGTCTTTTTTTG; C/EBPβ (reverse): TGTTCTTAATGCTTGAAACGGAA; Grp78 (forward): TTTTAATGATGCCCAACGCC; Grp78 (reverse): CTTCTCCCCCTCCCTCTTAT; GAPDH (forward): TCAAGAAGGTGGTGAAGCAG; GAPDH (reverse): GTGTCGCTGTTGAAGTCAGA, each at a final concentration of 300 nM. Reaction conditions were: 95° C. for 10 min; 95° C. for 15 sec and 60° C. for 1 min for 45 cycles. Melting curves were obtained as follows: 95° C. for 5 sec, 65° C. for 1 min, then gradual increase in temperature from 65° C. to 97° C., with a ramp rate of 0.11° C./s. The acquisition of the fluorescence signal was set as a single reading during the annealing step and as a continuous reading during the melting curve. Amplification efficiencies were calculated form dilution curves performed on 10 fold dilutions of one cDNA, which were used in the calculation of the GAPDH normalized expression ratios, applying the Pfaffl method.

XIV. Fluorescence Microscopy.

LN229 cells were transfected with various siRNAs, and mixed after 24 h with plasmid encoding cyan fluorescent protein (CFP) fused to human dynein light chain 3 (LC3) cDNA in the presence of Lipofectamine 2000 Transfection Reagent (Invitrogen). After 48 h, transfected cells were fixed in 4% paraformaldehyde for 15 min at 37° C., washed, and examined by fluorescence microscopy (Nikon, E600). The numbers of CFP-LC3 punctate dots per cell was determined from three independent experiments. A minimum of 50 CFP-LC3-positive cells assessed from at least ten random fields per sample were counted in triplicate per each condition.

XV. Analysis of Cell Viability and Autophagy.

Various cell types were seeded in triplicate onto 96-well plates at 4×10³ cells/well, treated with vehicle, Gamitrinib or 17-AAG (0-20 μM) for 12 h, and analyzed for changes in metabolic activity by an MTT assay. For determination of apoptosis, LN229 cells (1×106) were labeled for annexin V and propidium iodide (PI) (BD Biosciences), and analyzed by multiparametric flow cytometry (BD).

For analysis of tumor cell survival in 3D organotypic cultures, melanoma spheroids were prepared as previously described. Briefly, 50 μl aliquots of the various melanoma cell types (5×10⁴ cells/ml) were seeded onto 96-well plates coated with 1.5% agar (Difco, Sparks, Md.) in PBS. Spheroids were allowed to form for 72 h and then embedded in 100 μl of bovine collagen type I (Organogenesis, Canton, Mass.). Spheroids were overlaid with 100 μl of Tu2% growth medium and treated with inhibitors for 72 hours. Quantification of live vs. dead cells under the various conditions tested was performed by staining the melanoma spheroids after 72 h of treatment with calcein-AM (live) and Topro-3 (dead) (Invitrogen) for 1 h (data not shown).

Samples were imaged using a Leica TCS SP2 confocal microscope. For determination of autophagy, cells under the various conditions tested were harvested after 12 h, and analyzed with an antibody to dynein light chain-3 (LC3) by Western blotting.

XVI. Patient Samples.

A series of 217 consecutive patients surgically treated for non-small cell lung cancer (NSCLC) at Fondazione IRCCS Ca' Granda Hospital (Milan, Italy) between 2000 and 2004 was available for this study. This patient series included 154 cases of adenocarcinoma (AdCa) and 63 cases of squamous cell carcinoma (SCC) of the lung. Clinical outcome data were available for 167 patients (77%). NSCLC cases were staged according to the current TNM classification of malignant tumors (International Union Against Cancer, UICC, 7th edition, 2009). An informed consent was obtained from all patients enrolled, and the study was approved by an Institutional Review Board of the Fondazione IRCCS Ca' Granda, Milan, Italy. The follow-up period ranged from 0 to 132 months (average 55.2 months). At the last follow-up (January 2011), 94 patients were deceased for progression of NSCLC, whereas 73 patients were alive.

XVII. Tissue Microarray (TMA).

Representative tissue blocks from patients with various cancer diagnosis were used to construct the Cancer Universal TMA (CaU-TMA), and NSCLC TMAs (NSCLC-TMA). For the NSCLC-TMAs four cores of each patient were included in the blocks as well as 16 cores of non-neoplastic lung parenchyma. A total of 7 TMA blocks was then generated for the NSCLC series. For quality control, a 4-μm-thick section was cut from each TMA block, stained with H&E, and analyzed by immunohistochemistry.

XVIII. Immunohistochemistry.

The preclinical studies to test the efficacy and safety of Gamitrinib in a genetic mouse model of prostate cancer in immunocompetent TRAMP (Transgenic Adenocarcinoma of the Mouse Prostate) mice have been described (Kang et al., 2011). In this model, Gamitrinib was given systemically (5 mg/kg i.p. with the schedule 3 d on/2 d off) to TRAMP mice starting at 21.9 weeks of age (group 1) or 14.7 weeks of age (group 2), for 3 weeks or 5 weeks, respectively. In the control group, TRAMP mice of 24 weeks of age received vehicle. At the end of the experiment, animals in the vehicle or Gamitrinib groups were sacrificed and analyzed for localized and metastatic prostate cancer of adenocarcinoma or neuroendocrine histologic type (Kang et al., 2011). Of the TRAMP mice in the Gamitrinib treatment group, only one animal displayed a small residual area of prostatic adenocarcinoma in the dorso-lateral prostate (Kang et al., 2011).

For analysis of patient-derived or TRAMP tissues, slides were microwaved for 35 min in EDTA solution. Sections (4-μm thick) were cut from all TMA blocks and TRAMP-derived prostatic tumors and stained with a rabbit polyclonal antibody to Grp78 (1:1000) for 30 min at 22° C. IHC was performed using a Ventana BenchMark Ultra autostainer (Ventana Medical Systems), with the ultraView Universal DAB Detection Kit (Ventana) for detection of antibody reactivity. All slides were counterstained with hematoxylin. Immunoreactivity for Grp78 was evaluated by two pathologists (S.F. and S.B.) and independently scored for cytoplasmic localization. When discrepancies in scoring occurred, a consensus interpretation was reached after re-examination. The percentage of immunoreactive epithelial cells was determined and assigned to one of the following four categories: 0, (0%); 1, (1%-9%); 2, (10%-49%) and 3, (50%-100%). The intensity of immunostaining was scored as absent (0), mild (1), moderate (2) or strong (3). In the case of TMA, the two parameters were multiplied to generate a weighted score for each case that ranged from 0 to 9. Cases with weighted score 0 were defined as negative. Among AdCa samples in the series under investigation, 17 cases were negative for Grp78 expression, whereas 0 cases of SCC were Grp78-negative. The Grp78 score in positive cases was 2.3 (range: 0-9) for AdCa and 3.5 (range 0.5-9) for SCC.

XVIV. Statistical Analysis.

Data were analyzed using the two-sided unpaired t tests using a GraphPad software package (Prism 4.0) for Windows. For analysis of patient samples, groups were compared using the Wilcoxon or the Student's t tests as univariate statistics. For overall survival analysis the Kaplan-Meyer method was used. Patients negative for Grp78 immunoreactivity (score <0.25) were plotted separately from Grp78-positive cases (score ≥0.25) and the two-sided log-rank test was used to compare the two curves. Data are expressed as mean±SD or mean±SEM of multiple independent experiments. A p value of ≤0.05 was considered as statistically significant.

### Example 2: Gamitrinib Inhibits Glucose Consumption in Ln229 Glioblastoma Cells

For these experiments, the inventors used non-cytotoxic concentrations of Gamitrinib, which do not trigger mitochondrial permeability transition, but create non-lethal proteotoxic stress within the organelle, with accumulation of misfolded and insoluble proteins.

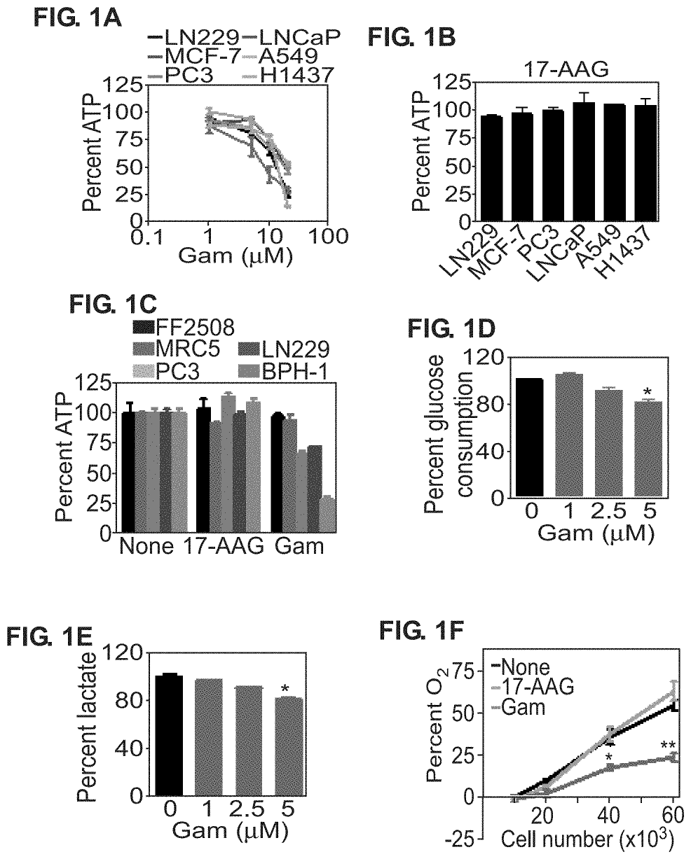

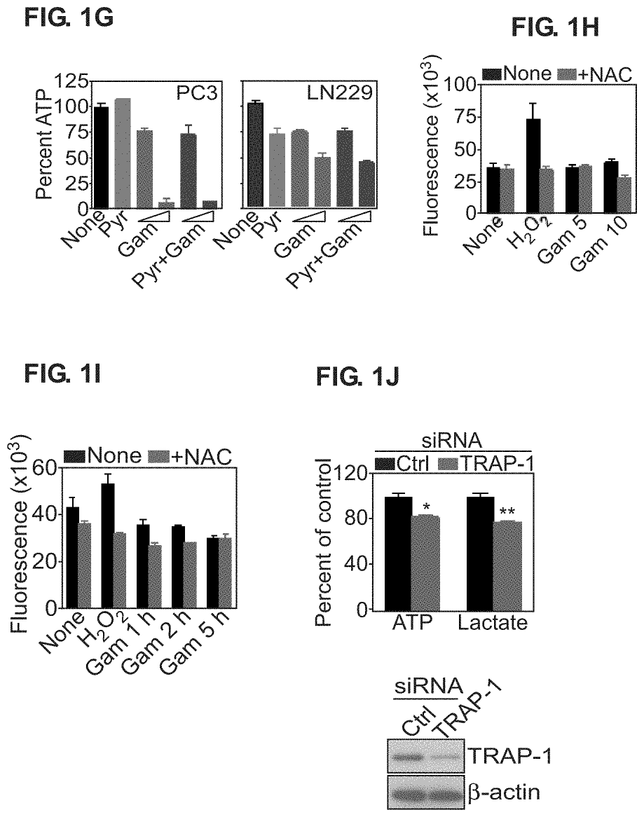

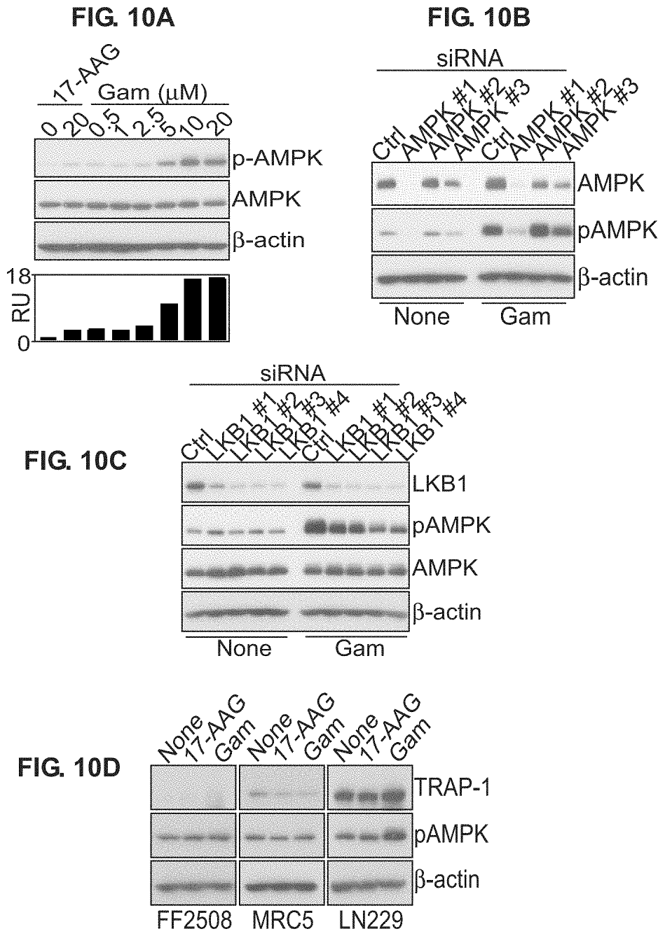

Under these conditions, Gamitrinib caused rapid (within 5 h), and concentration-dependent inhibition of ATP production in multiple tumor cell types (FIG. 1a). Conversely, 17-allylamino demethoxygeldanamycin (17-AAG), which inhibits Hsp90 ATPase activity in the cytsosol, but not mitochondria, had no effect on ATP production (FIG. 1b). The effect of Gamitrinib on bioenergetics was selective for tumor cells, as treatment of FF2508 or MRCS normal primary human fibroblasts did not change ATP production (FIG. 1c). Instead, Gamitrinib reduced glucose utilization (FIG. 1d), extracellular lactate levels (FIG. 1e), and oxygen consumption (FIG. 10, a marker of impaired oxidative phosphorylation, in tumor cells. Consistent with this finding, addition of exogenous sodium pyruvate failed to rescue ATP production in Gamitrinib-treated tumor cells (FIG. 1g). Whether at cytotoxic or non-cytotoxic concentrations (FIG. 1h), or for various time intervals (FIG. 1i), Gamitrinib had no effect on production of reactive oxygen species (ROS) in tumor cells.

As an independent approach, the inventors next knocked down by small interfering RNA (siRNA) the expression of TRAP-1 (FIG. 1j), one of the Hsp90 targets of Gamitrinib in mitochondria. TRAP-1 silencing mimicked the effect of Gamitrinib, and reduced ATP and lactate production in tumor cells (FIG. 1j). This effect was less pronounced than the response obtained with Gamitrinib, indicating the role of other mitochondrial chaperone(s), including Hsp90 in this pathway. A non-targeting siRNA had no effect (FIG. 1j).

### Example 3: Hsp90 Controls CYPD Protein Folding and Hexokinase-II Recruitment to Tumor Mitochondria

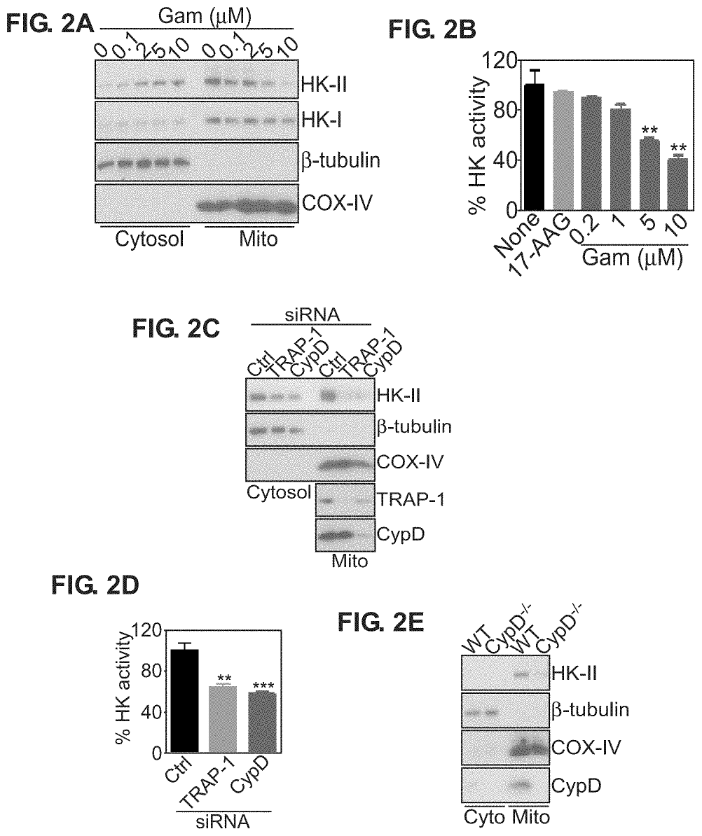

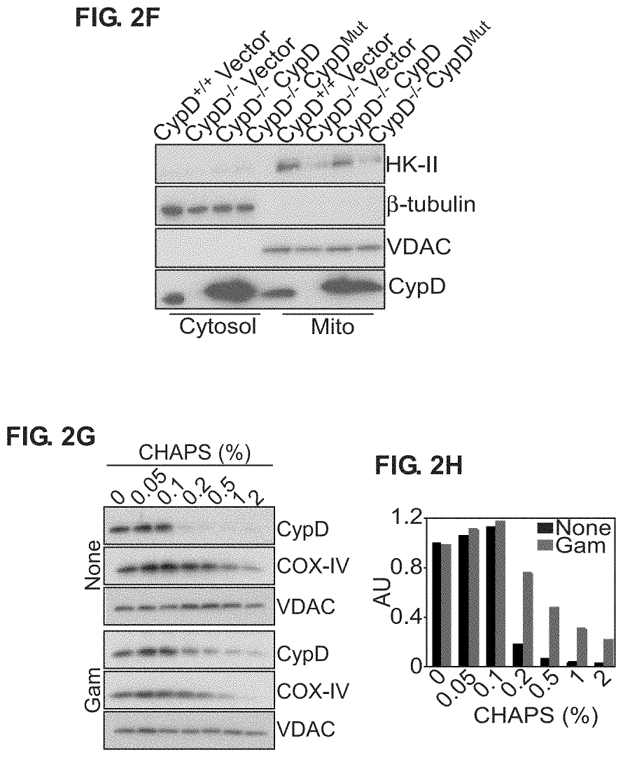

We next asked how mitochondrial Hsp90s controlled tumor bioenergetics. Mitochondrial proteotoxic stress imposed by Gamitrinib resulted in concentration-dependent release of hexokinase-II (HKII) from mitochondria, with its concomitant accumulation in the cytosol (FIG. 2a). Conversely, HK-I expression and subcellular localization were not affected (FIG. 2a). HK-II tethering to mitochondria is required for the first step of glycolysis, and to couple glucose metabolism to oxidative phosphorylation. Consistent with this model, Gamitrinib-treated tumor cells exhibited a decrease in hexokinase activity, whereas 17-AAG had no effect (FIG. 2b). siRNA knockdown of TRAP-1 gave similar results, with HK-II detachment from tumor mitochondria (FIG. 2c), and loss of hexokinase activity (FIG. 2d).

Mitochondrial Hsp90s have been shown to associate with the matrix peptidyl-prolyl isomerase (PPIase), CypD 9, a component of the permeability transition pore 11, and implicated in HK-II recruitment to the mitochondrial outer membrane 14. Here, siRNA knockdown of CypD released HK-II from mitochondria (FIG. 2c), and caused loss of hexokinase activity in tumor cells (FIG. 2d), quantitatively comparable to Gamitrinib treatment or TRAP-1 knockdown (FIG. 2c-d). Further, mitochondria isolated from CypD−/− mouse embryonic fibroblasts (MEFs) had reduced content of HK-II, compared to WT (CypD+/+) MEFs (FIG. 2e). Reconstitution of CypD−/− MEFs with a WT CypD cDNA restored binding of HK-II to mitochondria, whereas an H168Q CypD mutant defective in PPIase activity, or empty vector, had no effect (FIG. 20. The levels of outer membrane voltage-dependent anion channel (VDAC), which binds HK-II, were not affected (FIG. 20. We next asked whether CypD-mediated retention of HK-II to mitochondria involved chaperone-regulated protein folding, and we quantified changes in CypD solubility in response to Gamitrinib. In these experiments, inhibition of mitochondrial Hsp90s caused CypD to remain insoluble over a broad range of detergent concentrations, compared to untreated cultures (FIG. 2g). In contrast, the solubility of another mitochondrial protein, COX-IV was indistinguishable in control or Gamitrinib-treated cells, and VDAC remained insoluble at all detergent concentrations tested (FIG. 2g).

### Example 4: Regulation of Energy-Sensing Pathways by Mitochondrial Hsp90s

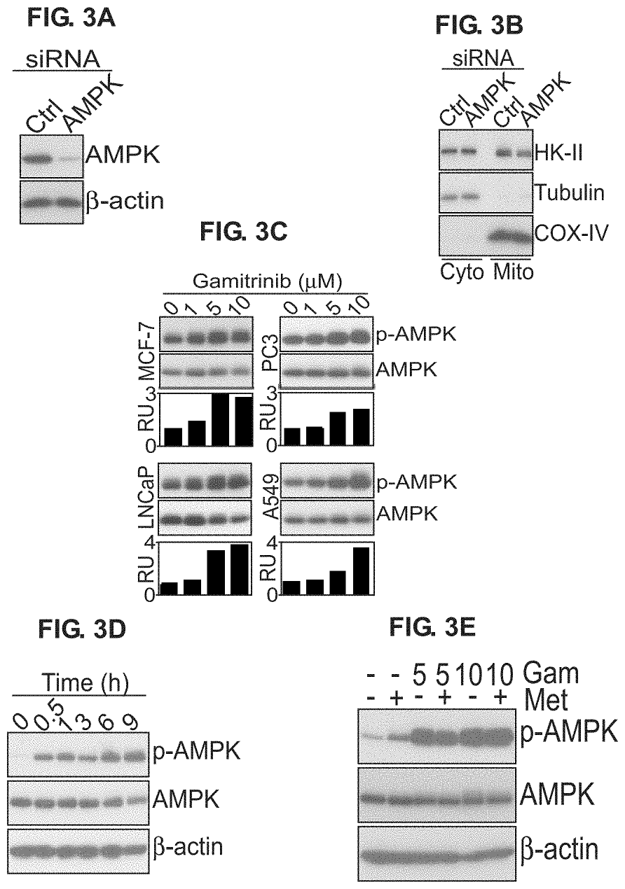

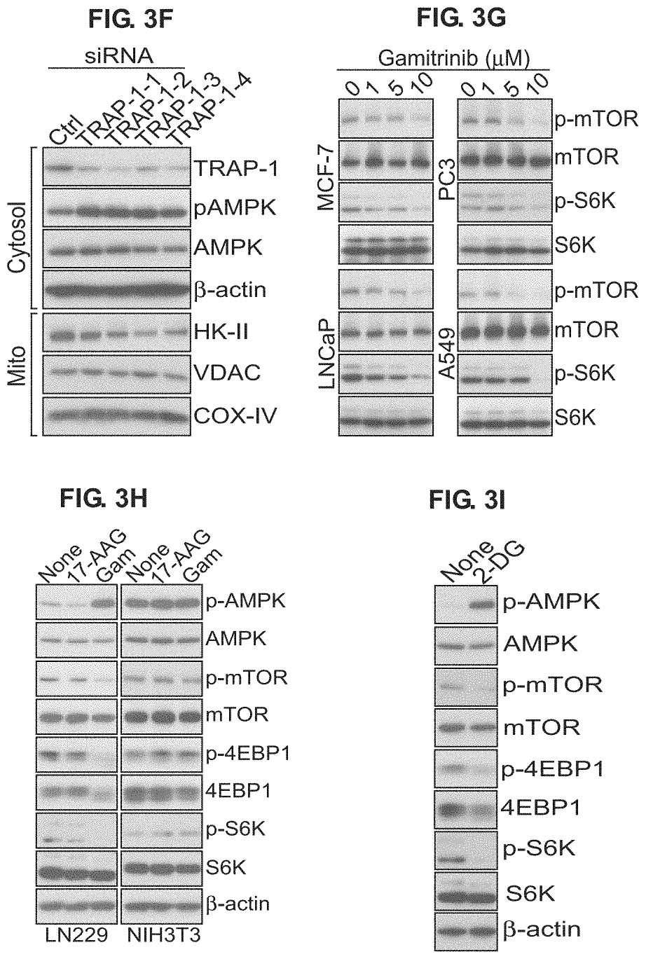

Detachment of HK-II from mitochondria decreases cellular energy production, and this is the main stimulus for activation of the energy-sensing AMP-activated kinase (AMPK). In a first set of experiments, siRNA silencing of AMPK did not significantly affect HK-II association with mitochondria in tumor cells (FIG. 3a), positioning AMPK downstream of HK-II-directed bioenergetics. Conversely tumor cells exposed to Gamitrinib, but not 17-AAG, exhibited concentration dependent phosphorylation of AMP-activated kinase (AMPK) (FIG. 3b, FIG. 10a), an energy-sensing molecule activated by nutrient deprivation. This AMPK activation occurred within 30 min of Gamitrinib treatment, remained sustained throughout 9 h (FIG. 3c), and was more quantitatively more prominent than that induced by metformin, a known AMPK inducer (FIG. 3d). Total AMPK levels were not affected (FIG. 3b-d), and the combination of metformin plus Gamitrinib did not further stimulate AMPK phosphorylation (FIG. 3d).

To validate these results, we next silenced the expression of AMPK, or its main upstream activator, the serine/threonine kinase LKB-1. Knockdown of AMPK (FIG. 10b) or LKB1 (FIG. 10c) using multiple, independent siRNA sequences inhibited Gamitrinib-induced AMPK phosphorylation (FIG. 10b-10c). This response was also selective for tumor cells, as FF2508 or MRCS primary human fibroblasts did not activate AMPK in response to Gamitrinib, and these cells were largely devoid of its target, TRAP-1, in mitochondria (FIG. 10d), in agreement with previous data. In complementary studies, we next silenced the expression of TRAP-1 using multiple, independent siRNA sequences (FIG. 3e). TRAP-1 knockdown in tumor cells resulted in strong phosphorylation of AMPK, and detachment of HK-II, but not VDAC or COX-IV, from mitochondria (FIG. 3e).

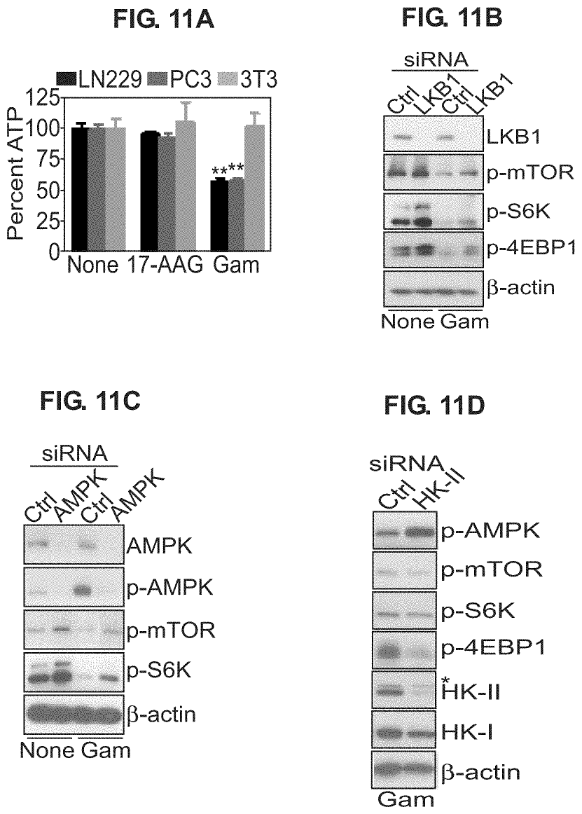

We next mapped the downstream consequences of impaired tumor bioenergetics. Consistent with current models of AMPK function, activation of this pathway by Gamitrinib inhibited the rapamycin-sensitive mammalian target of rapamycin complex-1 (mTORC1) in tumor cells, with concentration dependent suppression of phosphorylation of mTOR, and its downstream kinases 4EBP1 and p70S6 (FIG. 3f-3g). In contrast, inhibition of cytosolic Hsp90 with 17-AAG had no effect, and total mTORC1 protein content was unchanged (FIG. 3f-3g). The inhibition of mTORC1 under these conditions occurred selectively in tumor cells, as treatment of non-transformed NIH3T3 fibroblasts did not affect ATP production, or AMPK or mTORC1 phosphorylation (FIG. 3f-3g). As far as functional requirements for this pathway, siRNA knockdown of LKB1 (FIG. 11b), or AMPK (FIG. 11c), partially restored phosphorylation of mTOR, p70S6 and 4EBP1 in Gamitrinib treated tumor cells. Conversely, siRNA silencing of HK-II enhanced the effect of Gamitrinib, resulting in increased AMPK phosphorylation and mTORC1 inhibition (FIG. 11d). Consistent with a role of impaired tumor bioenergetics in mTORC1 modulation, tumor cells treated with the non-hydrolyzable glucose analog, 2-deoxy glucose (2-DG), which mimics energy starvation, exhibited AMPK activation and concomitant suppression of mTORC1 4EPB1 and p70S6 phosphorylation (FIG. 3h).

### Example 5: Mitochondrial Proteotoxic Stress Activates Pro-Survival Autophagy

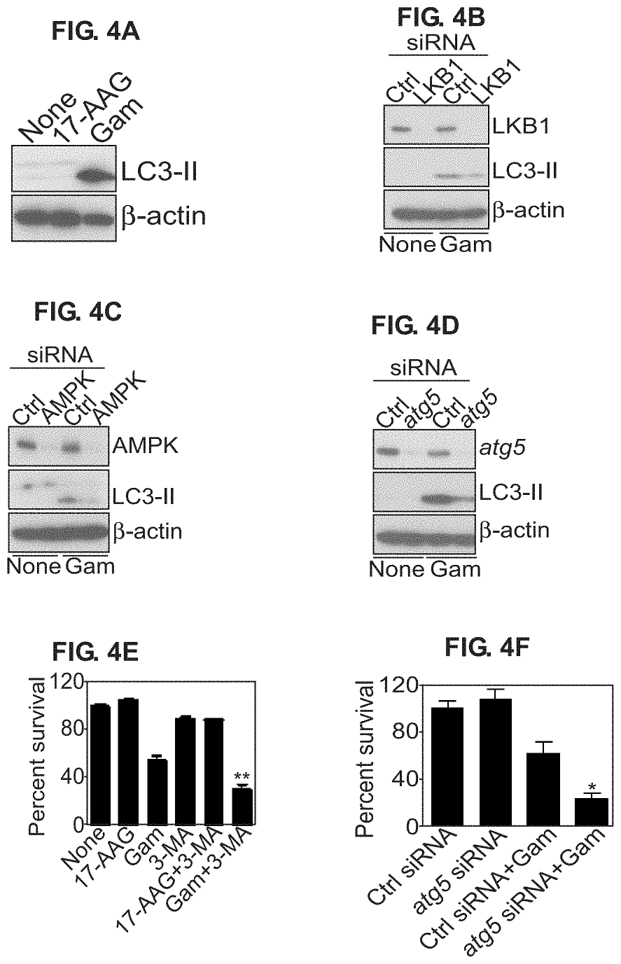

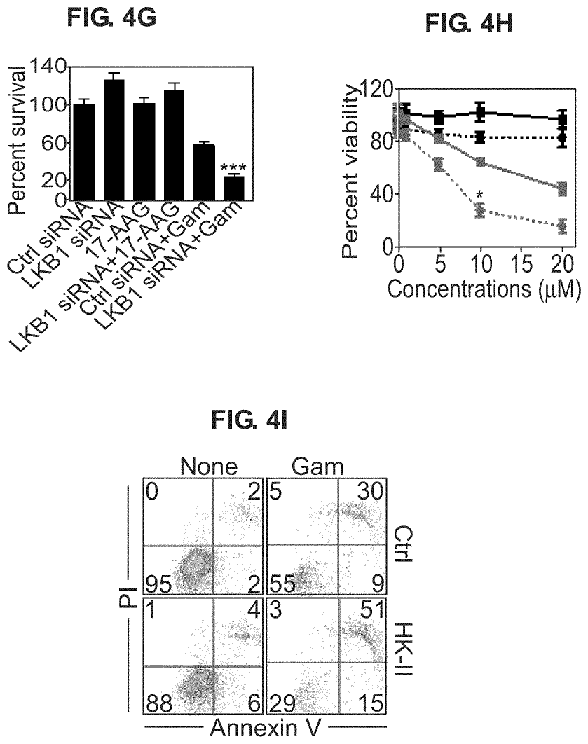

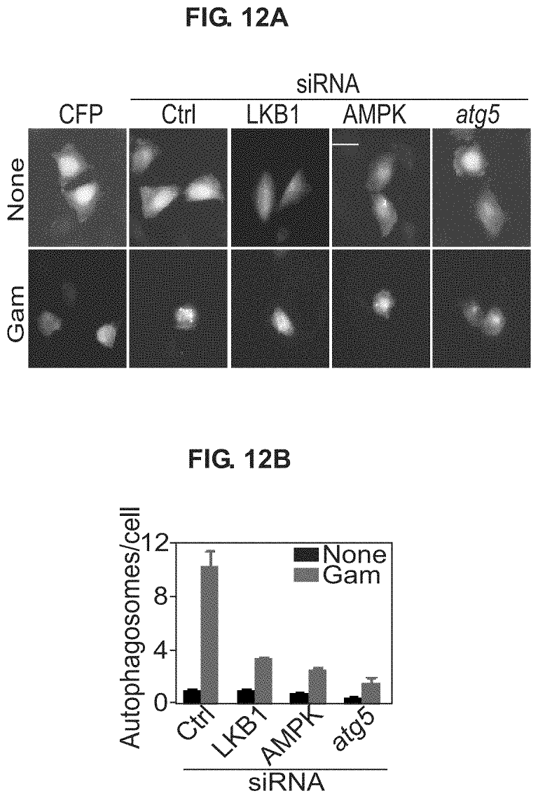

The functional implications of mTORC1 inhibition by mitochondrial proteotoxic stress were next investigated. Consistent with an inhibitory role of mTORC1 signaling on autophagy, and in agreement with recent observations, Gamitrinib strongly activated autophagy in tumor cells, with conversion of dynein light chain-3 (LC3-II) to a lipidated form (FIG. 4a), and appearance of a punctate fluorescence pattern of LC3-CFP staining in transfected cells (FIG. 12a-12b). Autophagy induction under these conditions required AMPK, as siRNA silencing of LKB1 (FIG. 4a), or AMPK (FIG. 4c), suppressed LC3-II conversion (FIG. 4b-4d), and autophagosome formation (FIG. 12a-12b) induced by Gamitrinib. As control, similar results were obtained with siRNA knockdown of the essential autophagy gene, atg5 (FIG. 4d), which mediates autophagosome expansion, in vivo.

We next asked whether autophagy activated under these conditions influenced tumor cell viability. In these experiments, inhibition of phagosome formation by 3-methyladenine (3-MA) (FIG. 4e), or siRNA knockdown of atg5 (FIG. 40, or LKB-1 (FIG. 4g), enhanced tumor cell killing mediated by sub-optimal concentrations of Gamitrinib. We also targeted the upstream initiation of this pathway, and siRNA silencing of HK-II (FIG. 11c) also potentiated tumor cell death over a broad range of Gamitrinib concentrations (FIG. 4h). Cell killing under these conditions had the characteristics of mitochondrial apoptosis with increased Annexin V labeling in Gamitrinib-treated HK-II silenced cells, compared to control transfectants (FIG. 4i). At variance with these data, the combination of 17-AAG plus 3-MA (FIG. 4e), or siRNA silencing of LKB1 (FIG. 4g), or HK-II (FIG. 4h), did not decrease tumor cell viability compared to each treatment alone.

### Example 6: Regulation of Metabolic Inter-Organelle Signaling by Mitochondrial Hsp90s

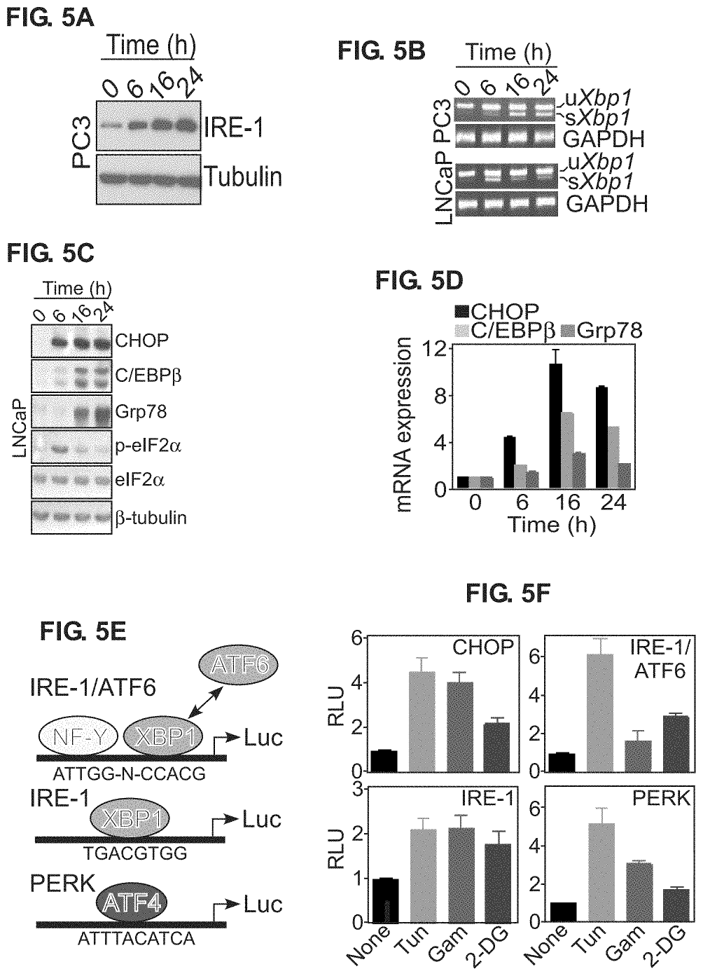

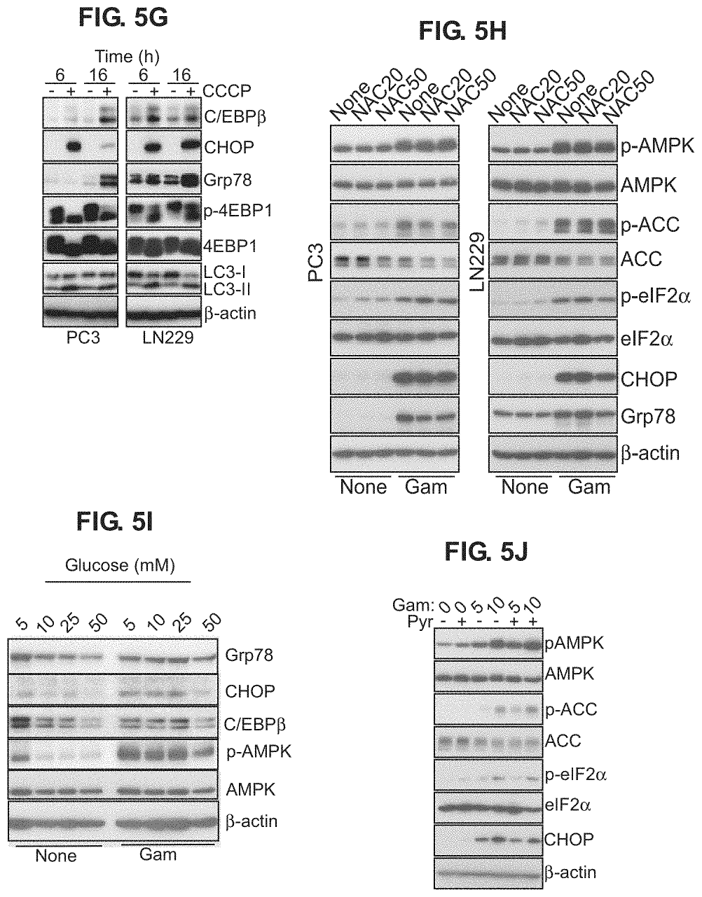

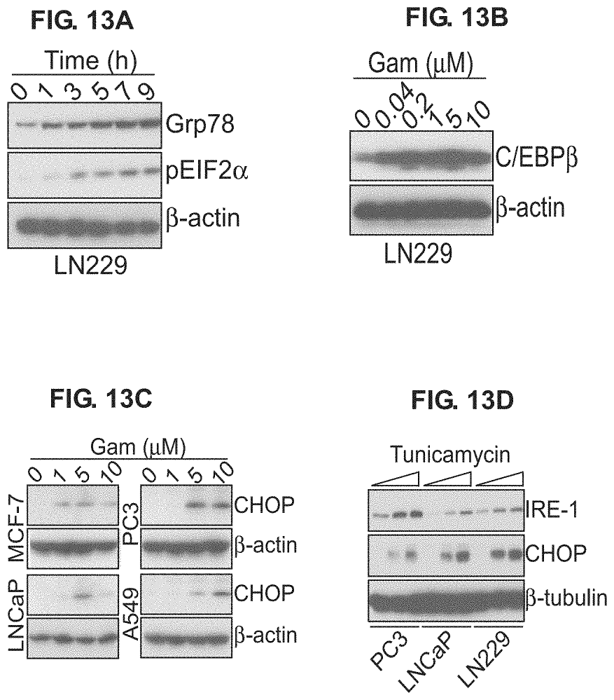

Defective mitochondrial bioenergetics may impair protein post-translational modifications in the ER, which in turn triggers an unfolded protein response (UPR). Within 1 h of Gamitrinib induced mitochondrial stress, tumor cells exhibited increased expression of Inositol-Requiring-1 (IRE-1) kinase (FIG. 5a), a pivotal ER stress sensor, and de novo mRNA splicing, i.e. activation, of its target, X-Box Protein-1, Xbp1 (FIG. 5b). Proteotoxic stress in mitochondria also stimulated the other ER UPR branches, with Activating Transcription Factor-6 (ATF-6)-mediated upregulation of the ER chaperone, Grp78 (FIG. 5c, FIG. 13a), and PKR-like endoplasmic reticulum kinase (PERK) induction of transcription factors, CCAAT-enhancer binding protein (C/EBPβ) and C/EBP homology protein (CHOP) 10 (FIG. 5c, FIG. 13b-13c). A transient phosphorylation of PERK-regulated eIF2α was also observed in cells exposed to Gamitrinib (FIG. 5c). This ER UPR was induced within 1 h of Gamitrinib treatment, and over a broad range of concentrations that did not affect mitochondrial integrity. As control, tumor cells treated with the ER stressor tunicamycin had comparable induction of IRE-1 and CHOP expression (FIG. 13d).

We next asked whether the ER UPR induced by mitochondrial stress involved de novo gene expression. Tumor cells treated with Gamitrinib, exhibited time-dependent upregulation of ER UPR markers, CHOP, C/EBPβ and Grp78 (FIG. 5d). In promoter analyses, Gamitrinib, 2-DG or tunicamycin all comparably induced transcriptional activation of pathway-specific response elements of all three ER UPR sensors, IRE-1, ATF-6 and PERK (FIG. 5e-5f). A minimal CHOP promoter region was also transcriptionally induced under these conditions (FIG. 5f).

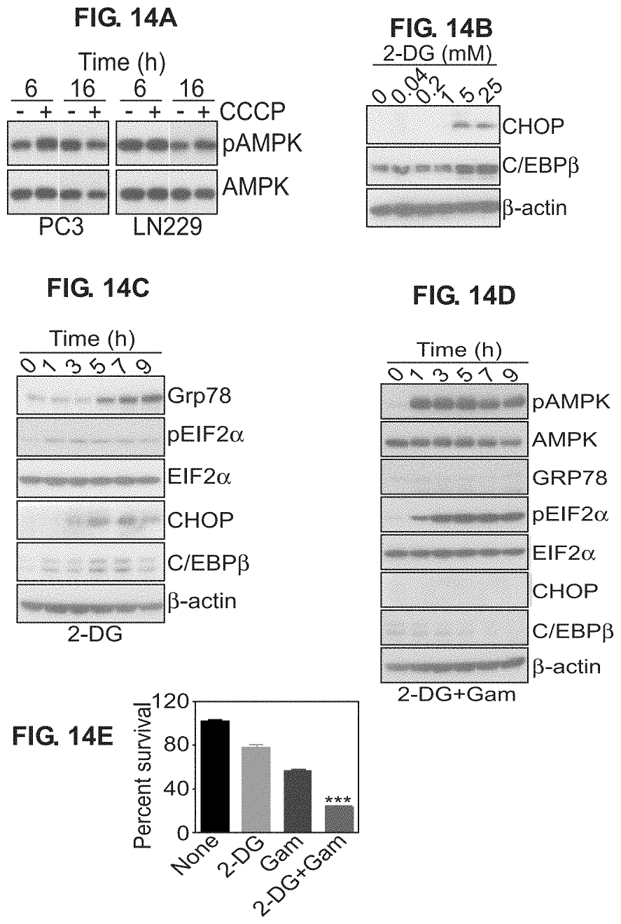

A mechanistic link between Gamitrinib-induced ER UPR and impaired mitochondrial bioenergetics was next investigated. First, we found that exposure of tumor cells to the uncoupler of mitochondrial membrane potential, carbonyl cyanide 3-chlorophenylhydrazone (CCCP), reproduced the multi-pathway response stimulated by Gamitrinib, with time-dependent phosphorylation of AMPK (FIG. 14a), upregulation of CHOP, C/EBPβ, and Grp78, inhibition of 4EBP1 phosphorylation, and stimulation of LC3-II conversion (FIG. 5g). In contrast, incubation of tumor cells with inhibitory concentrations of the ROS scavenger, N-acetyl-L-cysteine (NAC) did not affect Gamitrinib-induced phosphorylation of AMPK, its target acetyl-CoA carboxylase (ACC), or upregulation of ER stress markers (FIG. 5h). Second, energy deprivation, or impaired N-linked glycosylation, caused by 2-DG also mimicked the effect of Gamitrinib, resulting in concentration (FIG. 14b)- and time (FIG. 14c)-dependent upregulation of ER UPR in tumor cells. When combined with 2-DG, Gamitrinib maximally stimulated AMPK and eIF2α phosphorylation in tumor cells, which resulted in translational suppression of Grp78, CHOP or C/EBPβ (FIG. 14d), and increased tumor cell killing (FIG. 14e), compared to each agent alone.

In independent experiments, growing tumor cells in low glucose containing medium (5 mM) stimulated AMPK phosphorylation, and increased the expression of CHOP, C/EBPβ and Grp78 (FIG. 5i). Conversely, the addition of high glucose partially reversed this response, and attenuated the induction of ER UPR markers 9 and AMPK phosphorylation in response to Gamitrinib (FIG. 5i). Similar to ATP production (FIG. 1g), addition of exogenous sodium pyruvate did not affect the induction of phosphorylated AMPK or ER UPR markers in response to Gamitrinib (FIG. 5j).

### Example 7: Cytoprotective Role of ER UPR Induced by Gamitrinib

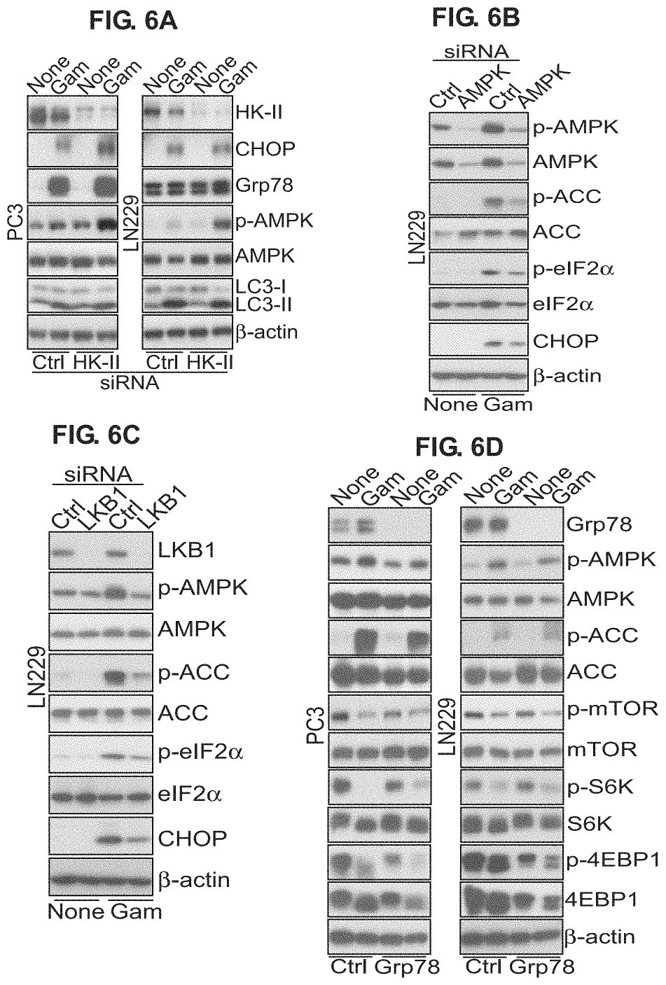

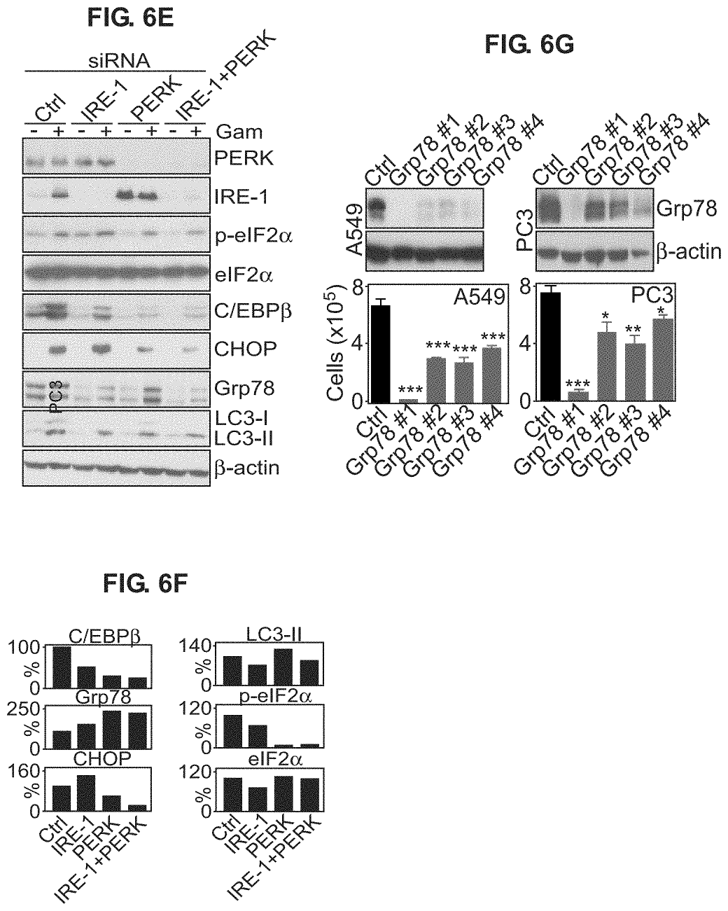

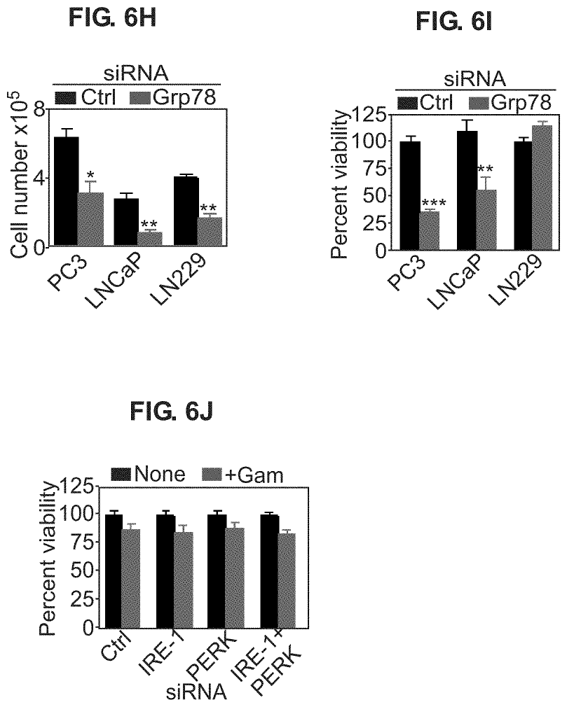

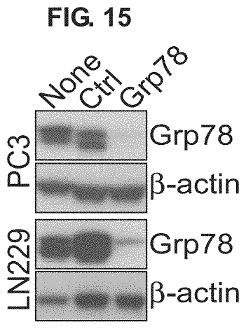

We next mapped the requirements of Gamitrinib-induced ER UPR. First, siRNA knockdown of HK-II was insufficient, alone, to upregulate the expression of CHOP or Grp78, promote AMPK phosphorylation, or stimulate LC3-II conversion in tumor cells (FIG. 6a). However, the combination of HK-II knockdown plus Gamitrinib further enhanced AMPK phosphorylation, ER UPR and autophagy in tumor cells (FIG. 6a). This pathway still depended on impaired tumor bioenergetics, as siRNA knockdown of AMPK (FIG. 6b), or LKB1 (FIG. 6c), attenuated Gamitrinib-induced phosphorylation of AMPK or its downstream target, acetyl-CoA carboxylase (ACC), and upregulation of ER UPR (FIG. 6b-6c). Conversely, siRNA silencing of the ER UPR effector, Grp78 (FIG. 6d, FIG. 15) did not affect AMPK pathway phosphorylation or the inhibition of mTORC1 signaling, i.e. 4EBP1 phosphorylation, induced by Gamitrinib, thus positioning Grp78 induction downstream of impaired ATP production (FIG. 1). Reciprocally, siRNA knockdown of the upstream ER stress sensor, IRE-1 did not significantly affect the induction of UPR markers by Gamitrinib (FIG. 6E). In contrast, knockdown of PERK, alone or in combination with IRE-1 silencing, inhibited the expression of CHOP and C/EBPβ, and abolished eIF2α phosphorylation induced by Gamitrinib, whereas GRP78 was not significantly affected, and no changes were observed in LC3 conversion (FIG. 6e). In all silencing experiments, a non-targeting siRNA was ineffective (FIG. 6a-6e).

The functional implications of ER UPR induced by mitochondrial proteotoxic stress were next investigated. Silencing of Grp78 using multiple, independent siRNA sequences inhibited tumor cell proliferation, compared to control siRNA transfectants (FIG. 60. Similar results were obtained after Grp78 knockdown in multiple tumor cell lines (FIG. 6g), indicating a general requirement of Grp78 for tumor cell proliferation. In addition, Grp78 knockdown decreased the viability of selected tumor cell types, including prostate cancer PC3 or LNCaP cells (FIG. 6h), suggesting a role of ER UPR in tumor cell survival. In contrast, IRE-1 or PERK knockdown, alone or in combination, did not reduce tumor cell viability in the presence or absence of Gamitrinib (FIG. 6i).

### Example 8: Chaperone-Regulated Bioenergetics Controls Tumor Maintenance

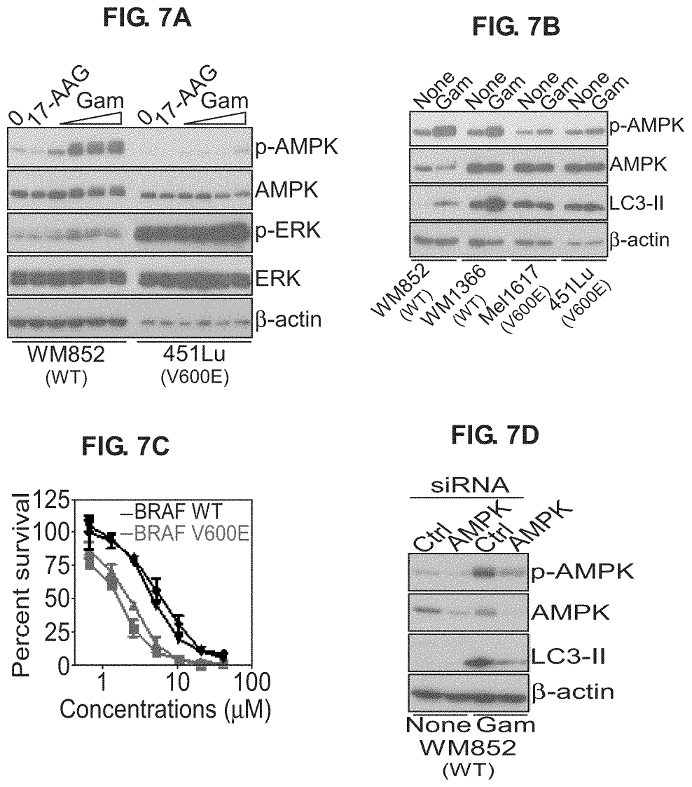

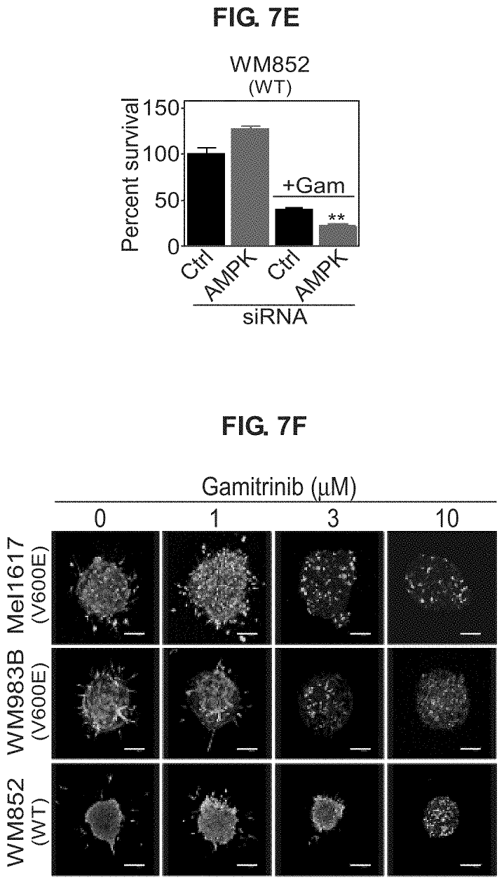

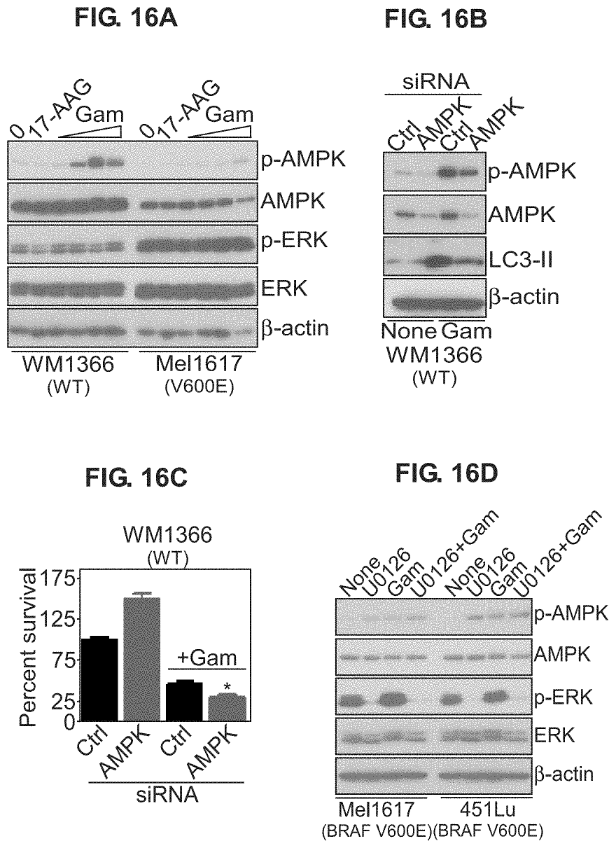

Next, we asked whether mitochondrial Hsp90-directed bioenergetics was important for tumor maintenance. We first looked at melanoma cells, where a V600E mutation of the BRAF oncogene results in ERK-mediated inhibitory phosphorylation of LKB-1, and suppression of AMPK activation. Accordingly, two BRAF mutant melanoma cells, which exhibited hyperphosphorylated ERK failed to activate AMPK after Gamitrinib treatment (FIG. 7a, FIG. 16a). Conversely, Gamitrinib induced AMPK phosphorylation in WT BRAF melanoma cells that had low levels of phosphorylated ERK (FIG. 7a, FIG. 16a). 17-AAG had no effect on AMPK activation in WT or mutant BRAF melanoma cells (FIG. 7a, FIG. 16a). Functionally, AMPK activation by mitochondrial stress stimulated autophagy in WT BRAF cells (FIG. 7b). Conversely, BRAF mutant cells did not adaptively increase autophagy in response to Gamitrinib (FIG. 7b), and were considerably more sensitive to mitochondrial apoptosis, compared to WT cultures (IC50 BRAF V600E: 1.95±0.21; IC50 BRAF WT: 6±1.4) (FIG. 7c). This differential susceptibility to cell death was reversed by siRNA knockdown of AMPK, which suppressed autophagy in WT BRAF cells (FIG. 7d, FIG. 16b), and enhanced their sensitivity to Gamitrinib-mediated killing (FIG. 7e, FIG. 16c). As control, an inhibitor of MEK (U0126) partially restored AMPK phosphorylation in mutant BRAF melanoma cells after Gamitrinib treatment (FIG. 16d). Next, we examined the impact of this pathway in a more disease-relevant model, and we reconstituted melanoma cell growth in 3D spheroids embedded in a collagen matrix. With this approach, we found that low concentrations of Gamitrinib (1-3 μM) efficiently killed mutant BRAF melanoma cells (FIG. 7f), whereas WT BRAF spheroids were resistant to cell death (FIG. 70. FIG. 7f are photomicrographs of melanoma cells with the indicated BRAF genotype grown as organotypic spheroids in 3D collagen-embedded matrices, incubated with the indicated concentrations of Gamitrinib, stained after 72 h with calcein-AM (live cells, light grey) and Topro-3 (dead cells, dark grey), and analyzed by confocal laser scanning microscopy. Representative images collected from one out of two independent determinations.

### Example 9: Mitochondrial Hsp90-Directed Bioenergetics Influences Tumor Outcome

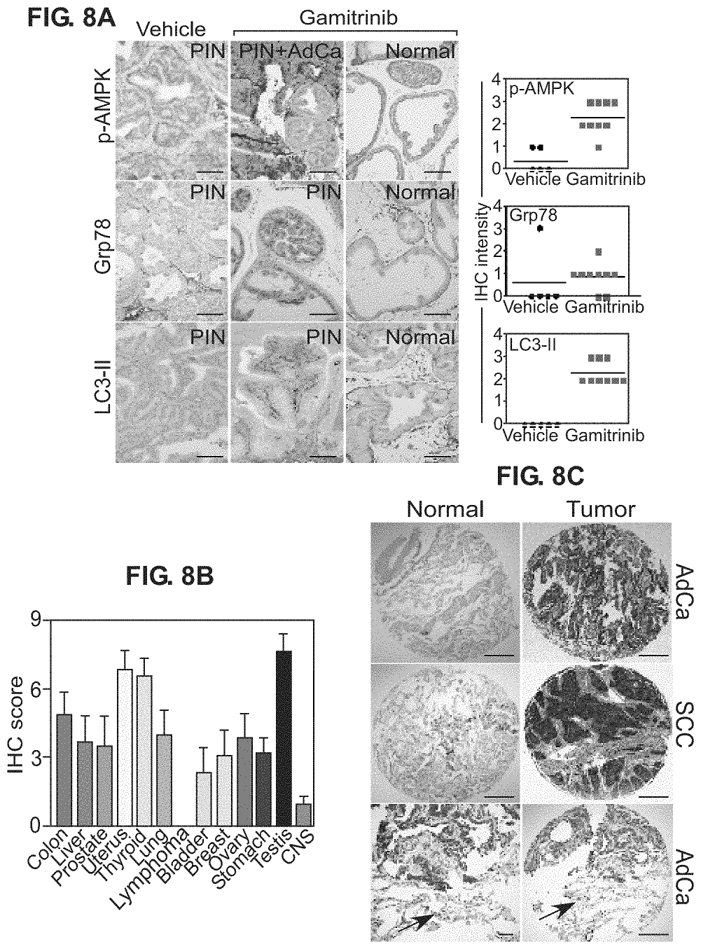

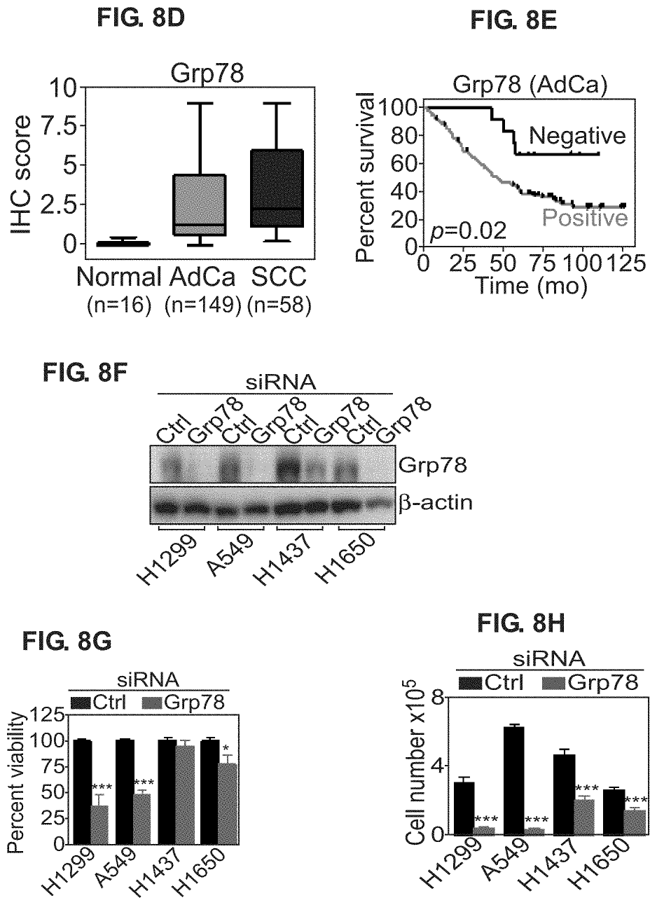

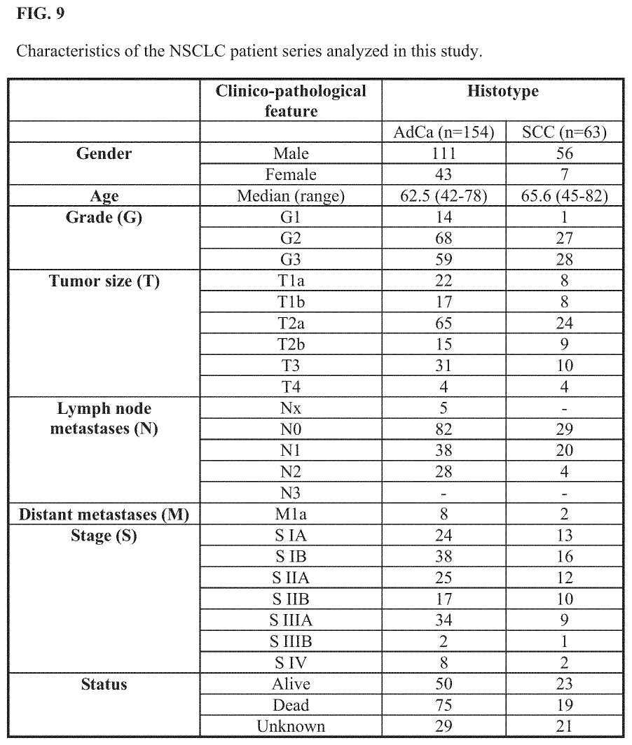

We next examined the role of the ER UPR activated by impaired bioenergetics in tumor progression, in vivo. we next examined a genetic model of prostate cancer in immunocompetent TRAMP (Transgenic Adenocarcinoma of the Mouse Prostate) mice treated systemically with Gamitrinib (Kang et al., 2011). In this model, Gamitrinib inhibited primary and metastatic prostate cancer growth, but did not affect Prostatic Intraepithelial Neoplasia (PIN) (Kang et al., 2011). Here, Gamitrinib treatment was associated with increased expression of phosphorylated AMPK, induction of autophagy, i.e. LC3 conversion, and upregulation of GRP78 in PIN lesions, but not in normal prostate (FIG. 8a). Prostate tissues from TRAMP mice treated with vehicle did not express these markers (FIG. 8a).

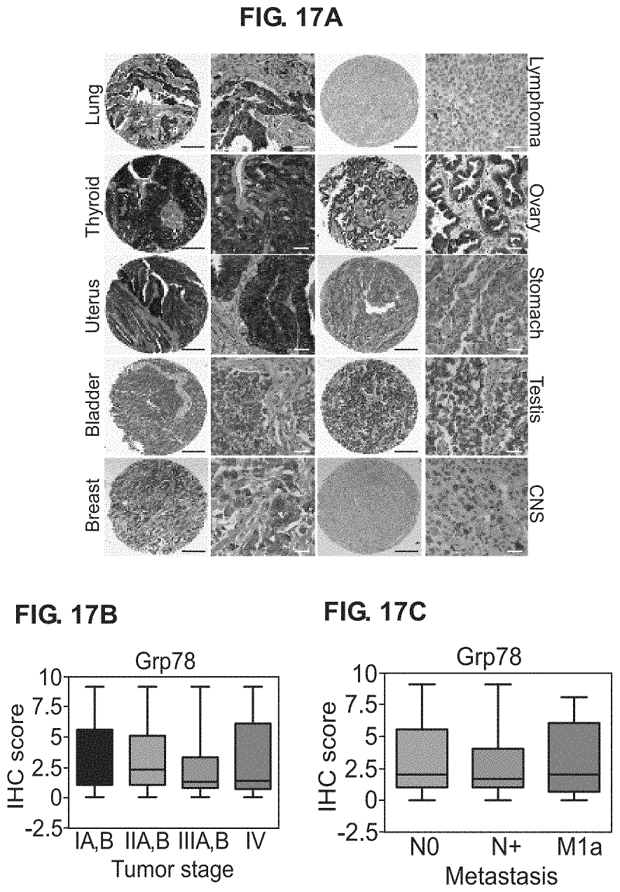

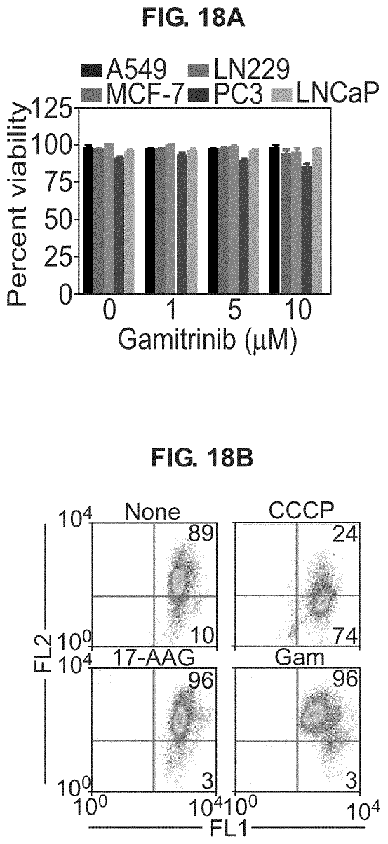

Except for lymphoma, Grp78 was strongly and uniformly upregulated in the tumor cell population of a large panel of genetically heterogeneous cancers, by tissue microarray analysis (FIG. 8b, FIG. 17a). Consistent with these findings, Grp78 was abundantly expressed in a cohort of non-small cell lung cancer patients (NSCLC, FIG. 9), with histologic variants of adenocarcinoma (AdCa) and squamous cell carcinoma (SCC) (FIG. 8c-8d), regardless of tumor stage (FIG. 17b), or lymph node metastasis (FIG. 17c). Conversely, Grp78 was undetectable in the normal epithelium of the lung (FIG. 8c-8d). When stratified for disease outcome, patients with lung AdCa expressing Grp78 had considerably shorter overall survival compared to NSCLC cases with low to undetectable Grp78 (FIG. 8e).

Based on these results, we asked whether deregulated expression of Grp78 influenced cell proliferation and/or survival in lung cancer cell types. Accordingly, siRNA silencing of Grp78 (FIG. 80, alone, was sufficient to induce loss of viability of H1299 or A549 lung cancer cell types, whereas H1457 and H1650 cells were only partially affected (FIG. 8g). Conversely, and similar to other tumor types (FIG. 6f-6g), silencing of Grp78 suppressed proliferation of all lung cancer cell types tested (FIG. 8h). A non-targeting siRNA had no effect (FIG. 8g-8h).

In this study, we have shown that compartmentalized mitochondrial Hsp90s are novel regulators of bioenergetics in tumor, but not normal cells. This pathway controls both glycolysis and oxidative phosphorylation, and is centered on chaperone-dependent retention of HK-II 13 to the organelle outer membrane. Interference with this mechanism by introducing non-lethal proteotoxic stress in mitochondria, a condition that may commonly occur during tumor growth, in vivo, resulted in acute decrease in ATP production, and activation of a global, nutrient-sensing signaling network, with phosphorylation of AMPK, inhibition of mTORC1, stimulation of autophagy, and induction of ER UPR. Functionally, this provides compensatory proliferative and cytoprotective mechanisms for tumor cells, and correlates with shortened overall survival in lung cancer patients.

CypD is the only known component of a mitochondrial permeability transition pore that is required for cell death triggered by certain stimuli, for instance oxidative stress. Chaperone-directed (re)folding of CypD may be a potential mechanism to preserve mitochondrial integrity, and antagonize apoptosis, selectively in tumor cells. Complete suppression of chaperone ATPase activity with Gamitrinib results in misfolding and aggregation of CypD, culminating with acute permeability transition and CypD-dependent cell death. Conversely, titrating the extent of chaperone inhibition using sub-optimal concentrations of Gamitrinib and shorter incubation times uncovered additional functional roles of this pathway, and, in particular, a mechanism of CypD conformation-dependent retention of HK-II to the outer mitochondrial membrane. In this context, detachment of HK-II after non-lethal mitochondrial proteotoxic stress is expected to lower an anti-apoptotic threshold maintained by growth factor-Akt signaling, but, even more importantly, to impair aerobic glycolysis, the main energy source for tumor cells. The inhibition of oxygen consumption observed here after Gamitrinib treatment, combined with the inability of exogenous pyruvate to restore ATP production under these conditions, suggest that organelle HSP90s may also contribute to oxidative phosphorylation. Loss of HK-II has been shown to shift tumor bioenergetics from aerobic glycolysis towards oxidative phosphorylation, potentially rendering tumor cells especially sensitive to the pathway of mitochondrial proteotoxicity described here.

Consistent with current models of energy-sensing homeostasis, loss of ATP production after mitochondrial proteotoxic stress resulted in downstream activation of an LKB1-AMPK signaling axis in tumor cells. There is compelling evidence that these molecules participate in tumor suppression, a concept also supported by the increasing instances of LKB1 inactivating mutations found in human cancers The LKB1-AMPK signaling axis participates in tumor suppression, and LKB1 is found mutated in certain human cancers. Here, however, activation of this pathway was exploited for tumor cell survival via stimulation of autophagy. Mechanistically, this may involve release of mTORC1 inhibition and/or direct AMPK activation of the autophagy initiation complex. AMPK phosphorylation is observed in hypoxic tumors deprived of nutrients, became prominently induced in PIN lesions of TRAMP mice treated with therapeutic concentrations of Gamitrinib, and downstream activation of autophagy has been identified as a driver of tumor maintenance, potentially at later stages of disease progression. In melanoma, where AMPK can be differentially activated depending on the mutational status of the BRAF oncogene, autophagy was a critical determinant of cell survival, making BRAF mutant cells especially sensitive to mitochondrial cell death, an observation with potential clinical relevance for melanoma patients who develop resistance to small molecule BRAF inhibitors. One of the consequences of acute decrease in ATP production is impaired protein posttranslational modifications, and in the case of mitochondrial Hsp90s, this resulted in a canonical ER UPR. This pathway was reproduced by pharmacologic uncoupling of mitochondrial membrane potential, in keeping with directional mitochondria-to-ER signaling, was partially reversed by high glucose, consistent with a causal role of defective ATP production in this process, and required LKB-1-AMPK activation as general effectors of bioenergetics signaling. In this context, emerging evidence supports a role of mitochondrial proteostasis influencing nuclear gene expression programs, potentially through the release of diffusible peptidyl mediators from mitochondria, or via inter-organelle signaling between physically juxtaposed ER and mitochondria regions.

The role of ER stress in cancer is complex, and prolonged activation of this pathway causes apoptosis, in part contributed by transcriptional modulation of Bcl-2 proteins. However, low level, chronic activation of ER stress may be beneficial for tumor growth, and, as suggested here, cooperate with AMPK-directed autophagy to antagonize apoptosis, reprogram gene expression to promote adaptation, and facilitate the acquisition of malignant traits, including metastatic potential, in vivo. Grp78 emerged here as a critical mediator of ER cytoprotection, sufficient as a biomarker to predict poor outcome in patients with lung AdCa, providing survival and/or proliferation signals for disparate tumor cell types. As to which of these two mechanisms becomes prevalent in Grp78-over-expressing cells may reflect intrinsic differences in anti-apoptotic threshold, including differential assembly of Bcl-2 homodimers, so that targeting Grp78 in cells with constitutive activation of multiple survival pathways, i.e. LN229, may only result in inhibition of cell proliferation. Regardless, the aberrant over-expression of Grp78 in disparate cancers observed here was an important predictor of unfavorable disease outcome, correlating with shortened overall survival in lung adenocarcinoma, similar to recent data obtained in prostate cancer. Regarding a potential role of other ER markers in disease outcome, high levels of eIF2α have been associated with improved survival in stage I, but not stage II-IV NSCLC patients.

As regulators of proteostasis, Hsp90s have long been recognized as nodal proteins, or “hubs” overseeing multiple signaling networks. The data presented here add new complexity to Hsp90-directed homeostasis, uncovering a role of the mitochondrial pools of these chaperones in controlling bioenergetics, and stress response signaling, selectively in tumors. The microenvironment of tumor growth, typically deprived of oxygen and nutrients, may produce a chronic degree of mitochondrial damage similar to the effect of non-cytotoxic exposure to Gamitrinib. Given their higher biosynthetic needs, it is also likely that actively proliferating tumors become especially prone to enhanced proteotoxic stress, including in the unique structural environment of mitochondria. Together, this may explain the selectivity of Hsp90 accumulation and function in tumor mitochondria, compared to normal tissues. The molecular basis for the differential recruitment of Hsp90s to tumor mitochondria has not been completely determined, and may be linked to oncogenic transformation. However, once in mitochondria, Hsp90s may be ideally poised to buffer organelle protoetoxic stress in general, and specifically control the (re)folding of CypD as a master switch of oxidative cell death and aerobic glycolysis. In turn, this prevents permeability pore opening, especially against oxidative stimuli, maintains ATP production via HK-II tethering, and connects to downstream survival mechanisms of autophagy (Yang et al., 2011) and GRP78 cytoprotection. Available data suggests that disparate tumors, regardless of their genetic heterogeneity, become nearly universally dependent on this adaptive pathway for growth and survival. This may offer broad therapeutic prospects, as subcellular targeting of mitochondrial Hsp90s is feasible, and, together with the tumor selectivity of this pathway, may disable global cellular networks of tumor adaptation and maintenance.

All patents, patent applications and other references, including US Provisional Patent Application Nos. 61/697,434 and 61/598,637, cited in this specification are hereby incorporated by reference in their entirety.

### REFERENCES

1. Mayer, M. P. Gymnastics of molecular chaperones. Mol. Cell 39, 321-331 (2010).
2. Taipale, M., Jarosz, D. F. & Lindquist, S. HSP90 at the hub of protein homeostasis: emerging mechanistic insights. Nat. Rev. Mol. Cell. Biol. 11, 515-528 (2010).
3. Trepel, J., Mollapour, M., Giaccone, G. & Neckers, L. Targeting the dynamic HSP90 complex in cancer. Nat. Rev. Cancer 10, 537-549 (2010).
4. Rodina, A. et al. Selective compounds define Hsp90 as a major inhibitor of apoptosis in small-cell lung cancer. Nat. Chem. Biol. 3, 498-507 (2007).
5. Richter, K., Reinstein, J. & Buchner, J. A Grp on the Hsp90 Mechanism. Mol. Cell 28, 177-179 (2007).
6. Leskovar, A., Wegele, H., Werbeck, N. D., Buchner, J. & Reinstein, J. The ATPase Cycle of the Mitochondrial Hsp90 Analog Trap1. J. Biol. Chem. 283, 11677-11688 (2008).
7. Haynes, C. M. & Ron, D. The mitochondrial UPR—protecting organelle protein homeostasis. J. Cell Sci. 123, 3849-3855 (2010).
8. Gandhi, S. et al. PINK1-associated Parkinson's disease is caused by neuronal vulnerability to calcium-induced cell death. Mol. Cell 33, 627-638 (2009).
9. Kang, B. H. et al. Regulation of tumor cell mitochondrial homeostasis by an organelle specific Hsp90 chaperone network. Cell 131, 257-270 (2007).
10. Siegelin, M. D. et al. Exploiting the mitochondrial unfolded protein response for cancer therapy in mice and human cells. J. Clin. Invest. 121, 1349-1360 (2011).
11. Green, D. R. & Kroemer, G. The pathophysiology of mitochondrial cell death. Science 305, 626-629 (2004).
12. Kang, B. H. et al. Combinatorial drug design targeting multiple cancer signaling networks controlled by mitochondrial Hsp90. J. Clin. Invest. 119, 454-464 (2009).
13. Vander Heiden, M. G., Cantley, L. C. & Thompson, C. B. Understanding the Warburg effect: the metabolic requirements of cell proliferation. Science 324, 1029-1033 (2009).
14. Machida, K., Ohta, Y. & Osada, H. Suppression of apoptosis by Cyclophilin D via stabilization of hexokinase II mitochondrial binding in cancer cells. J. Biol. Chem. 281, 14314-14320 (2006).
15. Mihaylova, M. M. & Shaw, R. J. The AMPK signalling pathway coordinates cell growth, autophagy and metabolism. Nat. Cell Biol. 13, 1016-1023 (2011).
16. Hezel, A. F. & Bardeesy, N. LKB1; linking cell structure and tumor suppression. Oncogene 27, 6908-6919 (2008).
17. Wullschleger, S., Loewith, R. & Hall, M. N. TOR signaling in growth and metabolism. Cell 124, 471-484 (2006).
18. Egan, D. F. et al. Phosphorylation of ULK1 (hATG1) by AMP-activated protein kinase connects energy sensing to mitophagy. Science 331, 456-461 (2011).
19. Kaufman, R. J. et al. The unfolded protein response in nutrient sensing and differentiation. Nat. Rev. Mol. Cell. Biol. 3, 411-421 (2002).
20. Hetz, C. & Glimcher, L. H. Fine-tuning of the unfolded protein response: Assembling the IRE1alpha interactome. Mol. Cell 35, 551-561 (2009).
21. Zheng, B. et al. Oncogenic B-RAF negatively regulates the tumor suppressor LKB1 to promote melanoma cell proliferation. Mol. Cell 33, 237-247 (2009).
22. Villanueva, J. et al. Acquired resistance to BRAF inhibitors mediated by a RAF kinase switch in melanoma can be overcome by cotargeting MEK and IGF-1R/PI3K. Cancer Cell 18, 683-695 (2010).
23. Majewski, N. et al. Hexokinase-mitochondria interaction mediated by Akt is required to inhibit apoptosis in the presence or absence of Bax and Bak. Mol. Cell 16, 819-830 (2004).
24. Yang, S. et al. Pancreatic cancers require autophagy for tumor growth. Genes Dev. 25, 717-729 (2011).
25. Nakagawa, T. et al. Cyclophilin D-dependent mitochondrial permeability transition regulates some necrotic but not apoptotic cell death. Nature 434, 652-658 (2005).
26. Baines, C. P. et al. Loss of cyclophilin D reveals a critical role for mitochondrial permeability transition in cell death. Nature 434, 658-662 (2005).
27. Robey, R. B. & Hay, N. Mitochondrial hexokinases, novel mediators of the antiapoptotic effects of growth factors and Akt. Oncogene 25, 4683-4696 (2006).
28. Inoki, K., Zhu, T. & Guan, K. L. TSC2 mediates cellular energy response to control cell growth and survival. Cell 115, 577-590 (2003).
29. Gwinn, D. M. et al. AMPK phosphorylation of raptor mediates a metabolic checkpoint. Mol. Cell 30, 214-226 (2008).
30. Laderoute, K. R. et al. 5′-AMP-activated protein kinase (AMPK) is induced by low oxygen and glucose deprivation conditions found in solid-tumor microenvironments. Mol. Cell. Biol. 26, 5336-5347 (2006).
31. Chhipa, R. R., Wu, Y. & Ip, C. AMPK-mediated autophagy is a survival mechanism in androgen-dependent prostate cancer cells subjected to androgen deprivation and hypoxia. Cell. Signal. 23, 1466-1472 (2011).
32. Pizzo, P. & Pozzan, T. Mitochondria-endoplasmic reticulum choreography: structure and signaling dynamics. Trends Cell Biol. 17, 511-517 (2007).
33. Tabas, I. & Ron, D. Integrating the mechanisms of apoptosis induced by endoplasmic reticulum stress. Nat. Cell Biol. 13, 184-190 (2011).
34. Ma, Y. & Hendershot, L. M. The role of the unfolded protein response in tumour development: friend or foe? Nat. Rev. Cancer 4, 966-977 (2004).
35. Dong, D. et al. A critical role for GRP78/BiP in the tumor microenvironment for neovascularization during tumor growth and metastasis. Cancer Res. 71, 2848-2857 (2011).
36. Balch, W. E., Morimoto, Dillin, A. & Kelly, J. W. Adapting proteostasis for disease intervention. Science 319, 916-919 (2008).
37. Whitesell, L. & Lindquist, S. L. HSP90 and the chaperoning of cancer. Nat. Rev. Cancer 5, 761-772 (2005).
38. Bouman, L. et al. Parkin is transcriptionally regulated by ATF4: evidence for an interconnection between mitochondrial stress and ER stress. Cell Death Differ. 18, 769-782 (2011).
39. Bruhat, A. et al. Amino Acids Control Mammalian Gene Transcription: Activating Transcription Factor 2 Is Essential for the Amino Acid Responsiveness of the CHOP Promoter. Mol. Cell. Biol. 20, 7192-7204 (2000).
40. Schinzel, A. C. et al. Cyclophilin D is a component of mitochondrial permeability transition and mediates neuronal cell death after focal cerebral ischemia. Proc. Natl. Acad. Sci. U.S.A. 102, 12005-12010 (2005).
41. Kang et al, Journal Clinical Investigation, 119(3):454-464 (March 2009)

1. A method of stimulating anti-tumor activity in a subject with cancer comprising:
administering to a subject in need thereof a low dosage of a composition comprising a molecule that inhibits Hsp90 linked to a mitochondria-penetrating moiety; and
administering to said subject an effective amount of an inhibitor of autophagy or glycolysis.


SEQUENCE LISTING (14 sequences)

SEQ ID NO: 1 - DNA, 35 bp, Artificial Sequence
  primer sequence
ctggttggatggcaagcaggttgtgttcggtcacg

SEQ ID NO: 2 - DNA, 35 bp, Artificial Sequence
  primer sequence
cgtgaccgaacacaacctgcttgccatccaaccag

SEQ ID NO: 3 - DNA, 22 bp, Artificial Sequence
  primer sequence
ttacgagagaaaactcatggcc

SEQ ID NO: 4 - DNA, 22 bp, Artificial Sequence
  primer sequence
gggtccaagttgtccagaatgc

SEQ ID NO: 5 - DNA, 23 bp, Artificial Sequence
  primer sequence
caaggtcatccatgacaactttg

SEQ ID NO: 6 - DNA, 22 bp, Artificial Sequence
  primer sequence
gtccaccaccctgttgctgtag

SEQ ID NO: 7 - DNA, 20 bp, Artificial Sequence
  primer sequence
tgttaaagatgagcgggtgg

SEQ ID NO: 8 - DNA, 22 bp, Artificial Sequence
  primer sequence
aggtgtggtgatgtatgaagat

SEQ ID NO: 9 - DNA, 26 bp, Artificial Sequence
  primer sequence
ttgttttgttttttggtctttttttg

SEQ ID NO: 10 - DNA, 23 bp, Artificial Sequence
  primer sequence
tgttcttaatgcttgaaacggaa

SEQ ID NO: 11 - DNA, 20 bp, Artificial Sequence
  primer sequence
ttttaatgatgcccaacgcc

SEQ ID NO: 12 - DNA, 20 bp, Artificial Sequence
  primer sequence
cttctccccctccctcttat

SEQ ID NO: 13 - DNA, 20 bp, Artificial Sequence
  primer sequence
tcaagaaggtggtgaagcag

SEQ ID NO: 14 - DNA, 20 bp, Artificial Sequence
  primer sequence
gtgtcgctgttgaagtcaga